(19) United States
(12) Patent Application Publication
(10) Pub. No.: US 2002/0012943 A1
(43) Pub. Date: Jan. 31, 2002

(54) ELECTROCHEMICAL PROBES FOR DETECTION OF MOLECULAR INTERACTIONS AND DRUG DISCOVERY

(76) Inventors: DANA M. FOWLKES, CHAPEL HILL, NC (US); H. HOLDEN THORP, CHAPEL HILL, NC (US)

Correspondence Address: MYERS BIGEL SIBLEY & SAJOVEC, PO BOX 37428, RALEIGH, NC, 27627, US

(21) Appl. No.: 09/019,679

(22) Filed: Feb. 6, 1998

Related U.S. Application Data
(60) Provisional application No. 60/036,919, filed on Feb. 6, 1997.
(60) Provisional application No. 60/059,049, filed on Sep. 16, 1997.

(51) Int. Cl. G01N 33/53

(52) U.S. Cl. 435/7.1

(57) ABSTRACT

This invention relates to methods and apparati for performing electrochemical analyses. The invention provides an electrochemical apparatus for performing potentio metric analyses for detecting specific binding between a first member of a biological binding pair immobilized on an electrode and a second member of a biological binding pair that is electrochemically labeled, in the presence of an electrochemical mediator. Methods for using the apparatus of the invention for performing binding and competition binding assays are provided. The invention also provides methods for performing high throughput screening assays for detecting inhibition of specific binding between the members of the biological binding pair for use in drug development, biochemical analysis and protein purification assays.

This application claims priority to U.S. Ser. No. 60/036,919, filed Feb. 6, 1997, and to U.S. Ser. No. 60/059,049, filed Sep. 16, 1997. The disclosures of each of these prior applications are specifically incorporated herein by reference.

### BACKGROUND OF THE INVENTION

1. Field of the Invention

The present invention relates to methods and apparati for performing electrochemical analyses that depend on specific binding between members of a biological binding pair. Specifically, the invention provides an electrochemical analysis apparatus for performing potentiometric analyses for detecting specific binding between a first member of a biological binding pair that is immobilized on an electrode with a second member of a biological binding pair that is electrochemically labeled, in the presence of an electrochemical mediator. Alternatively, the second member of the biological binding pair is linked to an electrochemical catalyst, preferably an enzyme and most preferably a redox enzyme, in the presence of an electrochemical mediator and a substrate for the electrochemical catalyst. In particular, apparati for performing cyclic voltammetric analyses of current produced over a range of applied voltages in the presence of electrochemically-labeled biologically active binding species are provided by the invention. Also provided are methods for using the apparatus of the invention for performing binding and competition binding assays, specifically competition binding assays using complex mixtures of biologically-active chemical species. The invention also provides methods for performing high throughput screening assays for detecting inhibition of specific binding between the members of the biological binding pair for use in drug development, biochemical analysis and protein purification assays.

2. Background of the Prior Art

U.S. Pat. No. 5,534,132, issued Jul. 9, 1996 to Vreeke et al. disclosed an electrode for use in detecting an affinity reaction.

U.S. Pat. No. 5,262,035, issued Nov. 16, 1993 to Gregg et al. disclosed a biosensor electrode using redox enzymes.

Vogt et al., 1965, Inorg. Chem. 4: 1157-1163 describes ruthenium-sulfur dioxide coordination compounds.

Ford et al., 1968, J. Amer. Chem. Soc. 90: 1187-1194 describes synthesis of pentaamine-ruthenium complexes of aromatic nitrogen heterocycles.

Yocum et al., 1982, Proc. Natl. Acad. Sci. USA 79: 7052-7055 describes preparation of a pentaamineruthenium derivative of horse heart ferricytochrome c.

Nocera et al., 1984,J. Amer. Chem. Soc. 106: 5145-5150 describes kinetics of intramolecular electron transfer from Ru^{II} to Fe^{III} in ruthenium-modified cytochrome c.

Devlin et al., 1990, Science 249: 404-406 describes random peptide libraries.

Hirai & Varmus, 1990, Molec. Cell. Biol. 10: 1307-1318 describes site-directed mutagenesis of src homology domain 3.

Lam et al., 1991, Nature (London) 354: 82-84 describes random peptide libraries.

Burrows et al., 1991, Eur. J. Biochem. 202: 543-549 describes direct electrochemistry of proteins.

Wanatabe-Fukunaga et al., 1992, Nature (London) 356: 314-317 describes fas as an apoptotic factor.

Salamon et al., 1992, Arch. Biochem. Biophys. 299: 193-198 describes direct electrochemistry of thioredoxins and glutathione.

Oldenburg et al., 1992, Proc. Natl. Acad. Sci. USA 89: 5393-5397 describes random peptide libraries.

Scott et al., 1992, Proc. Natl. Acad. Sci. USA 89: 5398-5402 describes random peptide libraries.

Hammer et al., 1992, J. Exp. Med. 176: 1007-1013 describes random peptide libraries.

Tsai & Weber, 1992, Anal. Chem. 64: 2897-2903 describes the influence of tyrosine on detection of copper-protein complexes.

O'Neil et al., 1992, Proteins: Structure, Function & Genetics 14: 509-515 describes GPIIb/IIIa antagonists obtained using phage display libraries.

Yu et al., 1992, Science 258: 1665-1668 describes src homology domain 3.

Dedman et al., 1993, J. Biol. Chem. 268: 23025-23030 describes random peptide libraries.

Chen et al., 1993, J. Amer. Chem. Soc. 115: 12591-12592 describes src homology domain 3-binding ligands using phage display libraries.

Kay et al., 1993, Gene 128: 59-65 describes random peptide libraries.

Hammer et al., 1993, Cell 74: 197-203 describes MHC-binding peptides.

Rozakis-Adcock et al., 1993, Nature (London) 363: 83-85 describes src SH3 domains.

Salamon et al., 1993, Proc. Natl. Acad. Sci. USA 90: 6420-6423 describes direct measurement of cyclic current-voltage responses using an electrode comprising an artificial lipid bilayer and integral membrane proteins.

Qureshi et al., 1993, Biomed. Chromatog. 7: 251-255 describes methods for detecting HPLC fractions containing biologically active peptides.

Okada et al., 1993, J. Biol. Chem. 268: 18070-18075 describes SH3-deleted src mutants.

Koivunen et al., 1993, J. Biol. Chem. 268: 20205-20210 describes phage display libraries.

Koivunen et al., 1993, J. Cell Biol. 124: 373-380 describes phage display libraries.

Hiramatsu et al., 1994, J. Biochem. 115: 584-589 describes electrochemical detection of polyamines.

Johnston et al., 1994, Inorg. Chem. 33: 6388-6390 describes rhenium-mediated electrocatalytic oxidation of DNA at indium tin-oxide electrodes as a method for voltammetric detection of DNA cleavage in solution.

Picksley et al., 1994, Oncogene 9: 2523-2529 describes binding between p53 and MDM 2.

Fong et al., 1994, Drug. Develop. Res. 33: 64-70 describes scanning whole cells with phage display libraries.

Yu et al., 1994, Cell 76: 933-945 describes src homology domain 3 fragments.

Uchida & Kawakishi, 1994, J. Biol. Chem. 269: 2405-2410 describes the identification of oxidized histidine at the active site of Cu, Zn superoxide dismutase.

Biachini & Wild, 1994, Toxicol. Lett. 72: 175-184 describes electrochemical detection of 7-methyldeoxyguanosine.

Cummings et al., 1994, J. Chromatog. B 653: 192-203 describes electrochemical detection of neuropeptide growth factor antagonists.

Mitton & Trevithick, 1994, Methods Enzymol. 233: 523-539 describes electrochemical detection of HPLC fractions for detecting antioxidant compounds in vertebrate lens.

Daniels & Lane, 1994, J. Molec. Biol. 243: 639-652 describes random peptide libraries.

Sparks et al., 1994, J. Biol. Chem. 269: 23853-23856 describes src homology domain 3 fragments.

Cheadle et al., 1994, J. Biol. Chem. 269: 24034-24039 describes src homology domain 3 fragments.

Rickles et al., 1994, EMBO J. 13: 5598-5604 describes src homology domain 3-binding ligands using phage display libraries.

Iwabuchi et al., 1994, Proc. Natl. Acad. Sci. USA 91: 6098-6102 describes p53 binding proteins.

Takahashi et al., 1994, Cell 76: 969-976 describes fas as an apoptotic factor.

Goodson et al., 1994, Proc. Natl. Acad. Sci. USA 91: 7129-7133 describes urokinase receptor antagonists obtained using phage display libraries.

Scharf et al., 1995, Biochem. Biophys. Res. Commun. 209: 1018-1025 describes electrochemical studies on nitrate reductase using a biosensor.

Abrams & Zhao, 1995, J. Biol. Chem. 270: 333-339 describes src homology domain 3.

Ivanenkov et al., 1995, J. Biol. Chem. 270: 14651-14658 describes random peptide libraries.

Takenaka et al., 1995, J. Biol. Chem. 270: 19839-19844 describes phage display libraries.

Martens et al., 1995, J. Biol. Chem. 270: 21129-21136 describes random peptide libraries.

Dyson & Murray, 1995, Proc. Natl. Acad. Sci. USA 92: 2194-2198 describes random peptide libraries.

Chen & Sudol, 1995, Proc. Natl. Acad. Sci. USA 92: 7819-7823 describes src homology domain 3.

Nagata & Golstein, 1995, Science 267:1449-1456 describes fas as an apoptotic factor.

Saksela et al., 1995, EMBO J. 14: 484-491 describes src homology domain 3.

Sudol et al., 1995, FEBS Lett. 369: 67-71 describes src homology domain 3.

Weng et al., 1995, Molec. Cell. Biol. 15: 5627-5634 describes src homology domain 3.

Rickles et al., 1995, Proc. Natl. Acad. Sci. USA 92: 10909-10913 describes src homology domain 3-binding ligands using phage display libraries.

Feng et al., 1995, Proc. Natl. Acad. Sci. USA 92: 12408-2415 describes src homology domain 3.

Gold et al., 1995, Ann. Rev. Biochem. 64: 763-797 describes aptamers.

Phizicky & Fields, 1995, Microbiol. Rev. 59: 94-123 describe methods for detecting and analyzing protein-protein interactions.

Adey & Kay, 1996, Gene 169: 133-134 describes random peptide libraries.

Sparks et al., 1996, Proc. Natl. Acad. Sci. USA 93: 1540-1543 describes src homology domain 3 fragments.

Yanofsky et al., 1996, Proc. Natl. Acad. Sci. USA 93: 7381-7386 describes high affinity interleukin type I antagonists obtained using recombinant peptide libraries.

Wrighton et al., 1996, Science 273:458-463 describes small peptides isolated from random peptide libraries as mimetics for erythropoietin.

Holmes et al., 1996, Science 274:2089-2091 describes src SH3 domains.

Fang et al., 1996, Biochem. Biophys. Res. Commun. 220: 53-56 describes trypsin inhibitors obtained using phage display libraries.

Hahne et al., 1996, Science 274: 1363-1366 describesfas as an apoptotic factor.

Chan et al., 1996, EMBO J. 15: 1045-1054 describes formin binding proteins having domains that functionally resemble src SH3 domains.

### SUMMARY OF THE INVENTION

The present invention provides methods and apparati for performing electrochemical analysis for detecting binding between a biological binding pair. These methods and apparati are useful for performing direct binding and competition binding experiments for detecting and analyzing compounds capable of inhibiting binding between the biological binding pair, thereby identifying compounds capable of interacting with biologically-active portions of the species comprising the biological binding pair. The methods of the invention are useful for performing rapid, high throughput screening of biologically active compounds for use as drugs that interact with one of the members of the biological binding pair and thereby interfere with or affect its biological function.

In a first aspect, the invention provides an apparatus for performing an electrochemical assay for detecting binding between members of a biological binding pair. The apparatus of the invention comprises the following components:

1. a first electrode, wherein the electrode comprises a conducting or semiconducting material, and wherein the electrode has a surface that is coated with a porous, hydrophilic, polymeric layer to which a first member of the biological binding pair is immobilized thereto;

2. a second, reference electrode comprising a conducting metal in contact with an aqueous electrolyte solution;

3. a third, auxiliary electrode comprising a conducting metal

wherein each of the electrodes is electrically connected to a potentiostat, and wherein the apparatus further comprises

4. a reaction chamber containing a solution of an electrolyte, wherein each of the electrodes is in electrochemical contact therewith, the solution further comprising

5. an electrochemical mediator comprising a chemical species capable of participating in a reduction/oxidation reaction with the electrodes, particularly the first electrode, under conditions whereby an electrical potential is applied to the electrodes, and wherein the solution further comprises

6. a second member of the biological binding pair, wherein said second member is electrochemically labeled with a chemical species capable of participating in a reduction/oxidation reaction with the electrochemical mediator under conditions whereby an electrical potential is applied to the electrodes.

In the use of this apparatus, a current is produced when an electrical potential is applied to the electrodes under conditions wherein the second member of the biological binding pair is bound to the first member of the biological binding pair.

In preferred embodiments, the electrochemical assay is cyclic voltammetry or chronoamperometry.

In a preferred embodiment, the first member of the biological binding pair is a receptor protein or ligand binding fragment thereof. In another preferred embodiment, the first member of the biological binding pair is an antibody protein or antigen binding fragment thereof. In yet another preferred embodiment, the first member of the biological binding pair is a first protein or fragment thereof that specifically binds to a second protein.

In preferred embodiments, the second member of the biological binging pair is a ligand, and antigen or a protein that binds to the first member of the biological binding pair immobilized on the first electrode of the apparatus of the invention. One of ordinary skill in the art will recognize the appropriate choice of first and second members of the biological binding pair (e.g., receptor/ligand, antigen/antibody, etc.).

In particularly preferred embodiments of the invention, the second member of the biological binding pair is a surrogate ligand for the first member of the biological binding pair, having an affinity of binding of from about 50 picomolar (pM) to about 0.5 mM, more preferably from about 1 nanomolar (nM) to about 100 micromolar (μM), and most preferably from about 10 nM to about 10 μM. Preferably said surrogate ligand is electrochemically labeled, more preferably with a ruthenium compound.

The apparatus of the invention also includes embodiments wherein the apparatus further comprises a multiplicity of each of the electrodes and reaction chambers of the invention, wherein each reaction chamber contains an electrolyte and is in electrochemical contact with one each of the three electrodes among the multiplicity of electrodes in the apparatus, and each of the electrodes in electrochemical contact with each reaction chamber is electrically connected to a potentiostat.

In preferred embodiments, the second member of the biological binding pair is electrochemically labeled with ruthenium. In preferred embodiments, the electrochemical mediator is a ruthenium compound. In particularly preferred embodiments, the ruthenium compound used as the electrochemical mediator or the electrochemical label is a pentaamineruthenium compound such as {Ru(NH₃)₅Cl}Cl, Ru(NH₃)₆³⁺ or Ru(NH₃)₅(H₂O)²⁺.

The invention also provides an electrode comprising a conducting or semiconducting material, wherein the electrode has a surface that is coated with a porous, hydrophilic, polymeric layer to which a first member of a biological binding pair is immobilized thereto, for use with the apparatus of the invention or for performing any other electrochemical assay.

The invention also provides a kit for preparing the first electrode of the apparatus of the invention. The kit provided by the invention comprises an electrode comprising a conducting or semi-conducting material, a first member of a biological binding pair, a reagent for preparing a porous, hydrophilic, polymeric layer on the surface of the electrode, and a reagent for immobilizing the first member of the biological binding pair within the porous, hydrophilic, polymeric layer on the surface of the electrode.

Accordingly, the invention also provides a method for preparing a first electrode of the apparatus of the invention, using the kit as provided herein or otherwise. These methods comprise the following steps:

a) providing an electrode comprising a conducting or semi-conducting material;

b) preparing a porous, hydrophilic, polymeric layer on the surface of the electrode; and

c) immobilizing a first member of the biological binding pair within the porous, hydrophilic, polymeric layer on the surface of the electrode.

The invention also provides a kit comprising a first electrode coated with an immobilized protein as described herein that is a first member of a biological binding pair, or alternatively the kit contains reagents for preparing said electrode wherein the reagents include the first member of the biological binding pair, preferably a protein, to be immobilized on the electrode, thus comprising an electrochemical target. Also provided as a component of these embodiments of the kits of the invention are at least one second member of the biological binding pair, preferably comprising a surrogate ligand having binding specificity for the first member of the biological binding pair characterized by a dissociation constant (K_{d}) of from about from about 50 picomolar (pM) to about 0.5 mM, more preferably from about 1 nanomolar (nM) to about 100 micromolar (μM), and most preferably from about 10 nM to about 10 μM, thus comprising an electrochemical probe. In certain embodiments of the kits of the invention, said second member of the biological binding pair is provided in an electrochemically labeled embodiment. In certain other embodiments of the kits of the invention, said second member of the biological binding pair is provided with reagents including an electrochemical label for preparing the electrochemically labeled embodiment by the user. The kit also provides an electrochemical mediator comprising a chemical species capable of participating in a reduction/oxidation reaction with the electrodes under conditions whereby an electrical potential is applied to the electrodes. Optionally and advantageously, the kit is also provided with an amount of the electrochemical mediator electrochemically matched to be useful according to the methods of the invention with the electrochemically-labeled probe. Additional and optional components of the kits of the invention include buffers, reagents and electrodes as described herein.

Methods of using the apparatus of the invention are also provided. In a first embodiment, a method for detecting binding of an electrochemically labeled second member of a biological binding pair with a first member of a biological binding pair immobilized on an electrode using an apparatus according to this aspect of the invention is provided. In this embodiment, the method comprises the steps of:

a) providing a first reaction chamber in electrochemical contact with each of the electrodes of the apparatus of the invention, wherein the first electrode comprises a first member of the biological binding pair immobilized thereto, and a second reaction chamber in electrochemical contact with each of the electrodes of the apparatus of the invention, wherein the first electrode comprises a first member of the biological binding pair immobilized thereto, each of the electrodes being electrically connected to a potentiostat;

wherein the first reaction chamber contains an electrochemical mediator of the apparatus of the invention and an electrochemically-labeled second member of the biological binding pair that specifically binds to the immobilized first member of the biological binding pair, and wherein the second reaction chamber comprises an electrochemical mediator of the apparatus of the invention and an electrochemically-labeled species that does not specifically bind to the immobilized first member of the biological binding pair; in other embodiments, the electrochemically-labeled second member of the biological binding pair that specifically binds to the immobilized first member of the biological binding pair is present in both the first and second reaction chambers, but the immobilized first member on the electrode in the second reaction chamber does not specifically bind the electrochemically-labeled second member. The method further comprises the steps of:

b) performing an electrochemical assay in each of the first and second reaction chambers of the apparatus of the invention to produce a current in the electrodes of the apparatus; and

c) comparing the current produced in the electrochemical assay in the first reaction chamber to the current produced in the electrochemical assay in the second reaction chamber

wherein binding of the electrochemically labeled second member of the biological binding pair with the immobilized first member of the biological binding pair is detected by the production of a larger current in the first reaction chamber than is produced in the second reaction chamber. Specific interaction between the members of the biological binding pair is detected by this comparison of the electrical current produced in each of the reaction chambers when an electrical potential is applied between the electrodes in each chamber. Specific binding of the first and second members of the biological binding pair in the first reaction chamber produces a higher current output in the first reaction chamber than is produced in the second reaction chamber, where there is no specific interaction between the second member of the biological binding pair and the unrelated species immobilized to the electrode in that chamber, or between the first member of the biological binding pair immobilized to the electrode in the second reaction chamber and the unrelated, electrochemically-labeled species contained in the second reaction chamber.

In a second embodiment of the methods of the invention is provided a method for identifying an inhibitor of binding of an electrochemically labeled second member of a biological binding pair with a first member of a biological binding pair immobilized on an electrode using an apparatus according to this aspect of the invention. In this embodiment, the method comprises the steps of:

a) providing a first reaction chamber in electrochemical contact with each of the electrodes of the apparatus of the invention, wherein the first electrode comprises a first member of the biological binding pair immobilized thereto, and a second reaction chamber in electrochemical contact with each of the electrodes of the apparatus of the invention, wherein the first electrode comprises a first member of the biological binding pair immobilized thereto, each of the electrodes being electrically connected to a potentiostat;

wherein each of the reaction chambers contains an electrochemical mediator of the apparatus of the invention and an electrochemically-labeled second member of the biological binding pair that specifically binds to the immobilized first member of the biological binding pair, and wherein the second reaction chamber further comprises an inhibitor of binding of a second member of the biological binding pair that specifically binds to the immobilized first member of the biological binding pair. The method further comprises the steps of:

b) performing an electrochemical assay in each of the first and second reaction chambers of the apparatus of the invention to produce a current in the electrodes of the apparatus; and

c) comparing the current produced in the electrochemical assay in the first reaction chamber to the current produced in the electrochemical assay in the second reaction chamber

wherein an inhibitor of binding of the electrochemically labeled second member of the biological binding pair with the immobilized first member of the biological binding pair is identified by the production of a larger current in the first reaction chamber than is produced in the second reaction chamber. Specific interaction between the members of the biological binding pair is detected by a comparison of the electrical current produced in each reaction chamber when an electrical potential is applied between the electrodes in the reaction chamber. The level and amount of current produced by specific binding of the first and second members of the biological binding pair in the reaction chamber is then compared with the level and amount of current produced in the chamber in the presence of an inhibitor of specific binding, and the difference related to the concentration and/or binding affinity of the inhibitor to the first member of the biological binding pair.

In yet a third embodiment of the methods of the invention is provided a method for screening a complex chemical mixture for an inhibitor of binding of an electrochemically labeled second member of a biological binding pair with a first member of a biological binding pair immobilized on an electrode using an apparatus of this aspect of the invention, the method comprising the steps of:

a) providing a first reaction chamber in electrochemical contact with each of the electrodes of the apparatus of the invention, wherein the first electrode comprises a first member of the biological binding pair immobilized thereto, and a second reaction chamber in electrochemical contact with each of the electrodes of the apparatus of the invention, wherein the first electrode comprises a first member of the biological binding pair immobilized thereto, each of the electrodes being electrically connected to a potentiostat;

wherein each of the reaction chambers contains an electrochemical mediator of the apparatus of the invention and an electrochemically-labeled second member of the biological binding pair that specifically binds to the immobilized first member of the biological binding pair, and wherein the second reaction chamber further comprises a portion of the complex mixture comprising an inhibitor of binding of the second member of the biological binding pair that specifically binds to the immobilized first member of the biological binding pair. The method further comprises the steps of:

b) performing an electrochemical assay in each of the first and second reaction chambers of the apparatus of the invention to produce a current in the electrodes of the apparatus; and

c) comparing the current produced in the electrochemical assay in the first reaction chamber to the current produced in the electrochemical assay in the second reaction chamber

wherein the complex mixture having an inhibitor of binding of the electrochemically labeled second member of the biological binding pair with the immobilized first member of the biological binding pair is identified by the production of a larger current in the first reaction chamber than is produced in the second reaction chamber. Specific interaction between the members of the biological binding pair is detected by a comparison of the electrical current produced in each reaction chamber when an electrical potential is applied between the electrodes in the chamber. The level and amount of current produced by specific binding of the first and second members of the biological binding pair in the reaction chamber is then compared with the level and amount of current produced in the chamber in the presence of a complex chemical mixture comprising an inhibitor of specific binding.

In an additional aspect of this embodiment of the invention, the method is used to isolate and identify an inhibitor of binding of the second member of the biological binding pair to the first member of the biological binding pair immobilized on the first electrode of the apparatus of the invention. In this embodiment, the method comprises the additional steps of:

d) chemically fractionating the complex mixture having an inhibitor of binding of the second member of the biological binding pair to the first member of the biological binding pair immobilized on the first electrode, to produce fractionated submixtures; and

e) performing steps (a) through (c) of the method on each of the fractionated submixtures to identify the submixtures that have an inhibitor of binding of the biological binding pair.

In this aspect, it will be recognized that steps (a) through (e) can be repeatedly performed on chemically fractionated submixtures to yield submixtures comprising increasingly purified preparations of the inhibitor. In preferred embodiments, the chemical fractionation includes chemical, biochemical, physical, and immunological methods for fractionation of chemical or biochemical species of inhibitor.

In preferred embodiments of each of the methods of the invention, the second member of a biological binding pair is an electrochemically labeled surrogate ligand characterized by a dissociation constant (K_{d}) for the first member of the biological binding pair of from about from about 50 picomolar (pM) to about 0.5 mM, more preferably from about 1 nanomolar (nM) to about 100 micromolar (μM), and most preferably from about 10 nM to about 10 μM.

In a second aspect of the invention is provided another apparatus for performing an electrochemical assay for detecting binding between members of a biological binding pair. In this aspect of the invention, the apparatus comprises the following components:

1. a first electrode, wherein the electrode comprises a conducting or semiconducting material, and wherein the electrode has a surface that is coated with a porous, hydrophilic, polymeric layer, wherein a first member of the biological binding pair and an electrochemical mediator comprising a chemical species capable of participating in a reduction/oxidation reaction with the electrodes under conditions whereby an electrical potential is applied to the electrodes, are each immobilized thereto,

2. a second, reference electrode comprising a conducting metal in contact with an aqueous electrolyte solution;

3. a third, auxiliary electrode comprising a conducting metal

wherein each of the electrodes is electrically connected to a potentiostat, and wherein the apparatus further comprises

4. a reaction chamber containing a solution of an electrolyte, wherein each of the electrodes is in electrochemical contact therewith, the solution further comprising

5. a second member of the biological binding pair, wherein said second member is electrochemically labeled with a chemical species capable of participating in a reduction/oxidation reaction with the electrochemical mediator under conditions whereby an electrical potential is applied to the electrodes.

In the use of this apparatus, a current is produced when an electrical potential is applied to the electrodes under conditions wherein the second member of the biological binding pair is bound to the first member of the biological binding pair.

In preferred embodiments, the electrochemical assay is cyclic voltammetry or chronoamperometry.

In a preferred embodiment, the first member of the biological binding pair is a receptor protein or ligand binding fragment thereof. In another preferred embodiment, the first member of the biological binding pair is an antibody protein or antigen binding fragment thereof. In yet another preferred embodiment, the first member of the biological binding pair is a first protein or fragment thereof that specifically binds to a second protein.

In preferred embodiments, the second member of the biological binging pair is a ligand, and antigen or a protein that binds to the first member of the biological binding pair immobilized on the first electrode of the apparatus of the invention. One of ordinary skill in the art will recognize the appropriate choice of first and second members of the biological binding pair (e.g., receptor/ligand, antigen/antibody, etc.).

In particularly preferred embodiments of the invention, the second member of the biological binding pair is a surrogate ligand for the first member of the biological binding pair, having an affinity of binding of from about 50 picomolar (pM) to about 0.5 mM, more preferably from about 1 nanomolar (nM) to about 100 micromolar (μM), and most preferably from about 10 nM to about 10 μM. Preferably said surrogate ligand is electrochemically labeled, more preferably with a ruthenium compound.

The apparatus of the invention also includes embodiments wherein the apparatus further comprises a multiplicity of each of the electrodes and reaction chambers of the invention, wherein each reaction chamber contains an electrolyte and is in electrochemical contact with one each of the three electrodes among the multiplicity of electrodes in the apparatus, and each of the electrodes in electrochemical contact with each reaction chamber is electrically connected to a potentiostat.

In preferred embodiments, the second member of the biological binding pair is electrochemically labeled with ruthenium. In preferred embodiments, the electrochemical mediator is a ruthenium compound or an osmium compound. In particularly preferred embodiments, the ruthenium compound used as the electrochemical mediator or the electrochemical label is a pentaamineruthenium compound such as {Ru(NH₃)₅Cl}Cl, Ru(NH₃)₆³⁺ or Ru(NH₃)₅(H₂O)²⁺. In preferred embodiments, the electrochemical mediator immobilized on the first electrode of the apparatus of the invention is an osmium bipyridine compound.

In the use of this embodiment of the invention, specific binding interactions between the members of the biological binding pair are detected by observation of an electrical current. Said electrical current is produced at an electrode potential sufficient to activate (oxidize or reduce) the immobilized electrochemical mediator and the electrochemical label attached to the second member of the biological binding pair. At said appropriate electrode potential, the oxidized (or reduced) electrochemical mediator is reduced by (oxidized by) the electrochemical label. The electrode potential permits cycles of oxidation/reduction of the electrochemical mediator/electrochemical label pair, thereby producing a current. In the practice of the invention, the amount of current produced by specific binding of the members of the biological binding pair is compared to the amount of current produced before addition of the second member of the biological binding pair, or to the amount of current produced upon addition of a known non-binding member (thereby providing a negative control). Specificity of binding is determined by comparison of the current to that generated in the presence of a known inhibitor of binding. Additional comparisons of the extent, capacity or rate of binding inhibition, activation or competition can be determined by analysis of the extent of produced current in the presence of putative inhibitors, competitors, activators or drug lead candidates, wherein specific details of the performance of such comparisons will be understood by those with skill in the art and are more fully disclosed below.

The invention also provides an electrode comprising a conducting or semiconducting material, wherein the electrode has a surface that is coated with a porous, hydrophilic, polymeric layer to which a first member of a biological binding pair and an electrochemical mediator comprising a chemical species capable of participating in a reduction/oxidation reaction with the electrodes under conditions whereby an electrical potential is applied to the electrodes, are each immobilized thereto, for use with the apparatus of the invention or for performing any other electrochemical assay.

The invention also provides a kit for preparing the first electrode of the apparatus of the invention. The kit provided by the invention comprises an electrode comprising a conducting or semi-conducting material, a first member of a biological binding pair, a reagent for preparing a porous, hydrophilic, polymeric layer on the surface of the electrode, an electrochemical mediator and a reagent for immobilizing the first member of the biological binding pair and the electrochemical mediator within the porous, hydrophilic, polymeric layer on the surface of the electrode.

Accordingly, the invention also provides a method for preparing a first electrode of the apparatus of the invention, using the kit as provided herein or otherwise. These methods comprise the following steps:

a) providing an electrode comprising a conducting or semi-conducting material;

b) preparing a porous, hydrophilic, polymeric layer on the surface of the electrode; and

c) immobilizing a first member of the biological binding pair and an electrochemical mediator within the porous, hydrophilic, polymeric layer on the surface of the electrode.

The invention also provides a kit comprising a first electrode coated with an immobilized protein as described herein that is a first member of a biological binding pair and an electrochemical mediator, or alternatively the kit contains reagents for preparing said electrode wherein the reagents include the first member of the biological binding pair, preferably a protein, to be immobilized on the electrode, thus comprising an electrochemical target, and an electrochemical mediator. Also provided as a component of these embodiments of the kits of the invention are at least one second member of the biological binding pair, preferably comprising a surrogate ligand having binding specificity for the first member of the biological binding pair characterized by a dissociation constant (K_{d}) of from about 50 picomolar (pM) to about 0.5 mM, more preferably from about 1 nanomolar (nM) to about 100 micromolar (μM), and most preferably from about 10 nM to about 10 μM, thus comprising an electrochemical probe. In certain embodiments of the kits of the invention, said second member of the biological binding pair is provided in an electrochemically labeled embodiment. In certain other embodiments of the kits of the invention, said second member of the biological binding pair is provided with reagents including an electrochemical label for preparing the electrochemically labeled embodiment by the user. Optionally and advantageously, the kit is also provided with an amount of the electrochemical mediator electrochemically matched to be useful according to the methods of the invention with the electrochemically-labeled probe. Additional and optional components of the kits of the invention include buffers, reagents and electrodes as described herein.

Methods of using the apparatus of the invention are also provided. In a first embodiment, a method for detecting binding of an electrochemically labeled second member of a biological binding pair with a first member of a biological binding pair immobilized on an electrode using an apparatus according to this aspect of the invention is provided. In this embodiment, the method comprises the steps of:

a) providing a first reaction chamber in electrochemical contact with each of the electrodes of the apparatus of the invention, wherein the first electrode comprises a first member of the biological binding pair and an electrochemical mediator comprising a chemical species capable of participating in a reduction/oxidation reaction with the electrode under conditions whereby an electrical potential is applied to the electrode immobilized thereto, and a second reaction chamber in electrochemical contact with each of the electrodes of the apparatus of the invention, wherein the first electrode comprises a first member of the biological binding pair and an electrochemical mediator comprising a chemical species capable of participating in a reduction/oxidation reaction with the electrode under conditions whereby an electrical potential is applied to the electrode immobilized thereto, each of the electrodes of the apparatus being electrically connected to a potentiostat;

wherein the first reaction chamber contains an electrochemically-labeled second member of the biological binding pair that specifically binds to the immobilized first member of the biological binding pair, and wherein the second reaction chamber comprises an electrochemically-labeled species that does not specifically bind to the immobilized first member of the biological binding pair; in other embodiments, the electrochemically-labeled second member of the biological binding pair that specifically binds to the immobilized first member of the biological binding pair is present in both the first and second reaction chambers, but the immobilized first member on the electrode in the second reaction chamber does not specifically bind the electrochemically-labeled second member. The method further comprises the steps of:

b) performing an electrochemical assay in each of the first and second reaction chambers of the apparatus of the invention to produce a current in the electrodes of the apparatus; and

c) comparing the current produced in the electrochemical assay in the first reaction chamber to the current produced in the electrochemical assay in the second reaction chamber

wherein binding of the electrochemically labeled second member of the biological binding pair with the immobilized first member of the biological binding pair is detected by the production of a larger current in the first reaction chamber than is produced in the second reaction chamber. Specific interaction between the members of the biological binding pair is detected by this comparison of the electrical current produced in each of the reaction chambers when an electrical potential is applied between the electrodes in each chamber. Specific binding of the first and second members of the biological binding pair in the first reaction chamber produces a higher current output in the first reaction chamber than is produced in the second reaction chamber, where there is no specific interaction between the second member of the biological binding pair and the unrelated species immobilized to the electrode in that chamber, or between the immobilized first member of the biological binding pair and the unrelated, electrochemically-labeled species contained in the second reaction chamber.

In a second embodiment of the methods of this aspect of the invention is provided a method for identifying an inhibitor of binding of an electrochemically labeled second member of a biological binding pair with a first member of a biological binding pair immobilized on an electrode using an apparatus according to the invention. In this embodiment, the method comprises the steps of:

a) providing a first reaction chamber in electrochemical contact with each of the electrodes of the apparatus of the invention, wherein the first electrode comprises a first member of the biological binding pair and an electrochemical mediator comprising a chemical species capable of participating in a reduction/oxidation reaction with the electrode under conditions whereby an electrical potential is applied to the electrode immobilized thereto, and a second reaction chamber in electrochemical contact with each of the electrodes of the apparatus of the invention, wherein the first electrode comprises a first member of the biological binding pair and an electrochemical mediator comprising a chemical species capable of participating in a reduction/oxidation reaction with the electrode under conditions whereby an electrical potential is applied to the electrode immobilized thereto, each of the electrodes of the apparatus being electrically connected to a potentiostat;

wherein each of the reaction chambers contains an electrochemically-labeled second member of the biological binding pair that specifically binds to the immobilized first member of the biological binding pair, and wherein the second reaction chamber further comprises an inhibitor of binding of the second member of the biological binding pair that specifically binds to the immobilized first member of the biological binding pair. The method further comprises the steps of:

b) performing an electrochemical assay in each of the first and second reaction chambers of the apparatus of the invention to produce a current in the electrodes of the apparatus; and

c) comparing the current produced in the electrochemical assay in the first reaction chamber to the current produced in the electrochemical assay in the second reaction chamber

wherein an inhibitor of binding of the electrochemically labeled second member of the biological binding pair with the immobilized first member of the biological binding pair is identified by the production of a larger current in the first reaction chamber than is produced in the second reaction chamber. Specific interaction between the members of the biological binding pair is detected by a comparison of the electrical current produced in each reaction chamber when an electrical potential is applied between the electrodes in the chamber. The level and amount of current produced by specific binding of the first and second members of the biological binding pair in the reaction chamber is then compared with the level and amount of current produced in the chamber in the presence of an inhibitor of specific binding, and the difference related to the concentration and/or binding affinity of the inhibitor to the first member of the biological binding pair.

In yet a third embodiment of the methods of this aspect of the invention is provided a method for screening a complex chemical mixture for an inhibitor of binding of an electrochemically labeled second member of a biological binding pair with a first member of a biological binding pair immobilized on an electrode using an apparatus of the invention. These methods comprise the steps of:

a) providing a first reaction chamber in electrochemical contact with each of the electrodes of the apparatus of the invention, wherein the first electrode comprises a first member of the biological binding pair and an electrochemical mediator comprising a chemical species capable of participating in a reduction/oxidation reaction with the electrode under conditions whereby an electrical potential is applied to the electrode immobilized thereto, and a second reaction chamber in electrochemical contact with each of the electrodes of the apparatus of the invention, wherein the first electrode comprises a first member of the biological binding pair and an electrochemical mediator comprising a chemical species capable of participating in a reduction/oxidation reaction with the electrode under conditions whereby an electrical potential is applied to the electrode immobilized thereto, each of the electrodes of the apparatus being electrically connected to a potentiostat;

wherein each of the reaction chambers contains an electrochemically-labeled second member of the biological binding pair that specifically binds to the immobilized first member of the biological binding pair, and wherein the second reaction chamber further comprises a portion of the complex mixture comprising an inhibitor of binding of a second member of the biological binding pair that specifically binds to the immobilized first member of the biological binding pair. The method further comprising the steps of:

b) performing an electrochemical assay in each of the first and second reaction chambers of the apparatus of the invention to produce a current in the electrodes of the apparatus; and

c) comparing the current produced in the electrochemical assay in the first reaction chamber to the current produced in the electrochemical assay in the second reaction chamber

wherein the complex mixture having an inhibitor of binding of the electrochemically labeled second member of the biological binding pair with the immobilized first member of the biological binding pair is identified by the production of a larger current in the first reaction chamber than is produced in the second reaction chamber. Specific interaction between the members of the biological binding pair is detected by a comparison of the electrical current produced in each reaction chamber when an electrical potential is applied between the electrodes in the chamber. The level and amount of current produced by specific binding of the first and second members of the biological binding pair in the reaction chamber is then compared with the level and amount of current produced in the chamber in the presence of a complex chemical mixture comprising an inhibitor of specific binding.

In an additional aspect of this embodiment of the invention, the method is used to isolate and identify an inhibitor of binding of the second member of the biological binding pair to the first member of the biological binding pair immobilized on the first electrode of the apparatus of the invention. In this embodiment, the method comprises the additional steps of:

d) chemically fractionating the complex mixture having an inhibitor of binding of the second member of the biological binding pair to the first member of the biological binding pair immobilized on the first electrode, to produce fractionated submixtures; and

e) performing steps (a) through (c) of the method on each of the fractionated submixtures to identify the submixtures that have an inhibitor of binding of the biological binding pair.

In this aspect, it will be recognized that steps (a) through (e) can be repeatedly performed on chemically fractionated submixtures to yield submixtures comprising increasingly purified preparations of the inhibitor. In preferred embodiments, the chemical fractionation includes chemical, biochemical, physical, and immunological methods for fractionation of chemical or biochemical species of inhibitor.

In preferred embodiments of each of the methods of the invention, the second member of the biological binding pair is an electrochemically labeled surrogate ligand characterized by a dissociation constant (K_{d}) for the first member of a biological binding pair of from about from about 50 picomolar (pM) to about 0.5 mM, more preferably from about 1 nanomolar (nM) to about 100 micromolar (μM), and most preferably from about 10 nM to about 10 μM.

In a third aspect of the invention is provided yet another apparatus for performing an electrochemical assay for detecting binding between members of a biological binding pair. In this aspect of the invention, the apparatus comprises the following components:

1. a first electrode, wherein the electrode comprises a conducting or semiconducting material, and wherein the electrode has a surface that is coated with a porous, hydrophilic, polymeric layer, wherein a first member of the biological binding pair and an electrochemical mediator comprising a chemical species capable of participating in a reduction/oxidation reaction with the electrodes under conditions whereby an electrical potential is applied to the electrodes, are each immobilized thereto,

2. a second, reference electrode comprising a conducting metal in contact with an aqueous electrolyte solution;

3. a third, auxiliary electrode comprising a conducting metal,

wherein each of the electrodes is electrically connected to a potentiostat, and wherein the apparatus further comprises

4. a reaction chamber containing a solution of an electrolyte, wherein each of the electrodes is in electrochemical contact therewith, the solution further comprising

5. a second member of the biological binding pair, wherein said second member is bound to an electrochemical catalyst capable of participating in a reduction/oxidation reaction with the electrochemical mediator under conditions whereby an electrical potential is applied to the electrode, wherein the electrolyte solution in the reaction chamber further comprises a substrate for the electrochemical catalyst.

In the use of this apparatus, a current is produced in the apparatus when an electrical potential is applied to the electrodes under conditions wherein the second member of the biological binding pair is bound to the first member of the biological binding pair in the presence of the substrate for the electrochemical catalyst bound to the second member of the biological binding pair.

In preferred embodiments, the electrochemical assay is cyclic voltammetry or chronoamperometry.

In a preferred embodiment, the first member of the biological binding pair is a receptor protein or ligand binding fragment thereof. In another preferred embodiment, the first member of the biological binding pair is an antibody protein or antigen binding fragment thereof. In yet another preferred embodiment, the first member of the biological binding pair is a first protein or fragment thereof that specifically binds to a second protein.

In preferred embodiments, the second member of the biological binging pair is a ligand, and antigen or a protein that binds to the first member of the biological binding pair immobilized on the first electrode of the apparatus of the invention. One of ordinary skill in the art will recognize the appropriate choice of first and second members of the biological binding pair (e.g., receptor/ligand, antigen/antibody, etc.).

In particularly preferred embodiments of the invention, the second member of the biological binding pair is a surrogate ligand for the first member of the biological binding pair, having an affinity of binding of from about 50 picomolar (pM) to about 0.5 mM, more preferably from about 1 nanomolar (nM) to about 100 micromolar (μM), and most preferably from about 10 nM to about 10 μM. Preferably said surrogate ligand is labeled with an electrochemical catalyst, preferably a redox enzyme such as horse radish peroxidase.

The apparatus of the invention also includes embodiments wherein the apparatus further comprises a multiplicity of each of the electrodes and reaction chambers of the invention, wherein each reaction chamber contains an electrolyte and is in electrochemical contact with one each of the three electrodes among the multiplicity of electrodes in the apparatus, and each of the electrodes in electrochemical contact with each reaction chamber is electrically connected to a potentiostat.

As provided in this aspect of the invention, the second member of the biological binding pair is labeled with an electrochemical catalyst. In preferred embodiments, the electrochemical catalyst is an enzyme, most preferably a redox enzyme capable of catalysis of its substrate to product by an oxidation/reduction mechanism wherein either functional groups on the enzyme of bound cofactors are involved in the oxidation/reduction cycle. In particularly preferred embodiments, the electrochemical catalyst is a peroxidase, for example horse radish peroxidase.

In preferred embodiments, the electrochemical mediator immobilized on the first electrode of the apparatus of the invention is an osmium compound, more preferably an osmium bipyridine compound.

In the use of this embodiment of the invention, specific binding interactions between the members of the biological binding pair are detected by observation of an electrical current. The apparatus of the invention comprises an electrode wherein an electrochemical mediator and the first member of the biological binding pair are both immobilized within the polymeric layer coating the electrode. The apparatus also comprises a second member of the biological binding pair chemically linked with a species, preferably an enzyme, that is capable of being oxidized or reduced by the immobilized mediator and also capable of catalytically oxidizing or reducing a third species present in the solution; in embodiments wherein the electrochemical catalyst is an enzyme, the third species is a substrate for the enzyme. This third species, however, cannot be directly oxidized or reduced by the immobilized mediator species present on the electrode. In the use of this embodiment of the invention, specific binding interactions between the members of the biological binding pair is detected by observation of an electrical current. Said electrical current is produced at an electrode potential sufficient to activate (oxidize or reduce) the immobilized electrochemical mediator and the electrochemical catalyst attached to the second member of the biological binding pair. At said appropriate electrode potential, the oxidized (or reduced) electrochemical mediator is reduced by (oxidized by) the electrochemical catalyst, thereby activating the catalyst for its substrate. As substrate is consumed, the electrode potential permits cycles of oxidation/reduction of the electrochemical mediator/electrochemical catalyst pair, thereby producing a current related to catalysis of the substrate by the electrochemical catalyst. In the practice of the invention, the amount of current produced by specific binding of the members of the biological binding pair is compared to the amount of current produced before addition of the second member of the biological binding pair, or to the amount of current produced upon addition of a known non-binding member (thereby providing a negative control). Specificity of binding is determined by comparison of the current to that generated in the presence of a known inhibitor of binding. Additional comparisons of the extent, capacity or rate of binding inhibition, activation or competition can be determined by analysis of the extent of produced current in the presence of putative inhibitors, competitors, activators or drug lead candidates, wherein specific details of the performance of such comparisons will be understood by those with skill in the art and are more fully disclosed below.

This aspect of the invention also provides an electrode comprising a conducting or semiconducting material, wherein the electrode has a surface that is coated with a porous, hydrophilic, polymeric layer to which a first member of a biological binding pair and an electrochemical mediator comprising a chemical species capable of participating in a reduction/oxidation reaction with the electrodes under conditions whereby an electrical potential is applied to the electrodes, are each immobilized thereto, for use with the apparatus of the invention or for performing any other electrochemical assay.

The invention also provides a kit for preparing the first electrode of the apparatus of the invention. The kit provided by the invention comprises an electrode comprising a conducting or semi-conducting material, a first member of a biological binding pair, a reagent for preparing a porous, hydrophilic, polymeric layer on the surface of the electrode, an electrochemical mediator and a reagent for immobilizing the first member of the biological binding pair and the electrochemical mediator within the porous, hydrophilic, polymeric layer on the surface of the electrode.

Accordingly, the invention also provides a method for preparing a first electrode of the apparatus of the invention, using the kit as provided herein or otherwise. These methods comprise the following steps:

a) providing an electrode comprising a conducting or semi-conducting material;

b) preparing a porous, hydrophilic, polymeric layer on the surface of the electrode; and

c) immobilizing a first member of the biological binding pair and an electrochemical mediator within the porous, hydrophilic, polymeric layer on the surface of the electrode.

The invention also provides a kit comprising a first electrode coated with an immobilized protein as described herein that is a first member of a biological binding pair and an electrochemical mediator, or alternatively the kit contains reagents for preparing said electrode wherein the reagents include the first member of the biological binding pair, preferably a protein, to be immobilized on the electrode, thus comprising an electrochemical target, and an electrochemical mediator. Also provided as a component of these embodiments of the kits of the invention are at least one second member of the biological binding pair, preferably comprising a surrogate ligand having binding specificity for the first member of the biological binding pair characterized by a dissociation constant (K_{d}) of from about 50 picomolar (pM) to about 0.5 mM, more preferably from about 1 nanomolar (nM) to about 100 micromolar (μM), and most preferably from about 10 nM to about 10 μM, thus comprising an electrochemical probe. In certain embodiments of the kits of the invention, said second member of the biological binding pair is provided linked to an electrochemical catalyst. In certain other embodiments of the kits of the invention, said second member of the biological binding pair is provided with reagents including an electrochemical catalyst for preparing the electrochemical catalyst-linked second member by the user. Optionally and advantageously, the kit is also provided with an amount of the electrochemical mediator electrochemically matched to be useful according to the methods of the invention with the electrochemical catalyst. Additional and optional components of the kits of the invention include buffers, reagents and electrodes as described herein.

Methods of using this apparatus of the invention are also provided. In a first embodiment, a method for detecting binding of an electrochemically labeled second member of a biological binding pair with a first member of a biological binding pair immobilized on an electrode using an apparatus according to this aspect of the invention is provided. In this embodiment, the method comprises the steps of:

a) providing a first reaction chamber in electrochemical contact with each of the electrodes of the apparatus of the invention, wherein the first electrode comprises a first member of the biological binding pair and an electrochemical mediator comprising a chemical species capable of participating in a reduction/oxidation reaction with the electrode under conditions whereby an electrical potential is applied to the electrode immobilized thereto, and a second reaction chamber in electrochemical contact with each of the electrodes of the apparatus of the invention, wherein the first electrode comprises a first member of the biological binding pair and an electrochemical mediator comprising a chemical species capable of participating in a reduction/oxidation reaction with the electrode under conditions whereby an electrical potential is applied to the electrode immobilized thereto, each of the electrodes of the apparatus being electrically connected to a potentiostat;

wherein the first reaction chamber contains a second member of the biological binding pair that specifically binds to the immobilized first member of the biological binding pair, bound to an electrochemical catalyst, and wherein the second reaction chamber contains a species bound to an electrochemical catalyst that does not specifically bind to the immobilized first member of the biological binding pair, and each reaction chamber further contains a substrate for the electrochemical catalyst; in other embodiments, the second member of the biological binding pair that specifically binds to the immobilized first member of the biological binding pair bound to an electrochemical catalyst is in both the first and second reaction chambers, but the immobilized first member on the electrode in the second reaction chamber does not specifically bind the electrochemical catalyst-linked second member. The method further comprises the steps of:

b) performing an electrochemical assay in each of the first and second reaction chambers of the apparatus of the invention to produce a current in the electrodes of the apparatus; and

c) comparing the current produced in the electrochemical assay in the first reaction chamber to the current produced in the electrochemical assay in the second reaction chamber

wherein binding of the electrochemically labeled second member of the biological binding pair with the immobilized first member of the biological binding pair is detected by the production of a larger current in the first reaction chamber than is produced in the second reaction chamber. Specific interaction between the members of the biological binding pair is detected by this comparison of the electrical current produced in each of the reaction chambers when an electrical potential is applied between the electrodes in each chamber. Specific binding of the first and second members of the biological binding pair in the first reaction chamber produces a higher current output in the first reaction chamber than is produced in the second reaction chamber, where there is no specific interaction between the second member of the biological binding pair and the unrelated species immobilized to the electrode in that chamber, or between the immobilized first member of the biological binding pair and the unrelated, electrochemically-labeled species contained in the second reaction chamber.

In a second embodiment of the methods of this aspect of the invention is provided a method for identifying an inhibitor of binding of an electrochemically labeled second member of a biological binding pair with a first member of a biological binding pair immobilized on an electrode using an apparatus according to the invention. In this embodiment, the method comprises the steps of:

a) providing a first reaction chamber in electrochemical contact with each of the electrodes of the apparatus of the invention, wherein the first electrode comprises a first member of the biological binding pair and an electrochemical mediator comprising a chemical species capable of participating in a reduction/oxidation reaction with the electrode under conditions whereby an electrical potential is applied to the electrode immobilized thereto, and a second reaction chamber in electrochemical contact with each of the electrodes of the apparatus of the invention, wherein the first electrode comprises a first member of the biological binding pair and an electrochemical mediator comprising a chemical species capable of participating in a reduction/oxidation reaction with the electrode under conditions whereby an electrical potential is applied to the electrode immobilized thereto, each of the electrodes of the apparatus being electrically connected to a potentiostat;

wherein each of the reaction chambers contains a second member of the biological binding pair that specifically binds to the immobilized first member of the biological binding pair, bound to an electrochemical catalyst, and a substrate for the electrochemical catalyst, and wherein the second reaction chamber further contains an inhibitor of binding of the second member of the biological binding pair that specifically binds to the immobilized first member of the biological binding pair. The method further comprises the steps of:

b) performing an electrochemical assay in each of the first and second reaction chambers of the apparatus of the invention to produce a current in the electrodes of the apparatus; and

c) comparing the current produced in the electrochemical assay in the first reaction chamber to the current produced in the electrochemical assay in the second reaction chamber

wherein an inhibitor of binding of the electrochemically labeled second member of the biological binding pair with the immobilized first member of the biological binding pair is identified by the production of a larger current in the first reaction chamber than is produced in the second reaction chamber. Specific interaction between the members of the biological binding pair is detected by a comparison of the electrical current produced in the reaction chamber when an electrical potential is applied between the electrodes in the chamber. The level and amount of current produced by specific binding of the first and second members of the biological binding pair in the reaction chamber is then compared with the level and amount of current produced in the chamber in the presence of an inhibitor of specific binding.

In yet a third embodiment of the methods of this aspect of the invention is provided a method for screening a complex chemical mixture for an inhibitor of binding of an electrochemically labeled second member of a biological binding pair with a first member of a biological binding pair immobilized on an electrode using an apparatus of the invention. These methods comprise the steps of:

a) providing a first reaction chamber in electrochemical contact with each of the electrodes of the apparatus of the invention, wherein the first electrode comprises a first member of the biological binding pair and an electrochemical mediator comprising a chemical species capable of participating in a reduction/oxidation reaction with the electrode under conditions whereby an electrical potential is applied to the electrode immobilized thereto, and a second reaction chamber in electrochemical contact with each of the electrodes of the apparatus of the invention, wherein the first electrode comprises the first member of the biological binding pair and an electrochemical mediator comprising a chemical species capable of participating in a reduction/oxidation reaction with the electrode under conditions whereby an electrical potential is applied to the electrode immobilized thereto, each of the electrodes being electrically connected to a potentiostat;

wherein each of the reaction chambers contains a substrate for the electrochemical catalyst and a second member of the biological binding pair that specifically binds to the immobilized first member of the biological binding pair, bound to an electrochemical catalyst, and wherein the second reaction chamber further comprises a portion of the complex mixture comprising an inhibitor of binding of the second member of the biological binding pair that specifically binds to the immobilized first member of the biological binding pair. The method further comprises the steps of:

b) performing an electrochemical assay in each of the first and second reaction chambers of the apparatus of the invention to produce a current in the electrodes of the apparatus; and

c) comparing the current produced in the electrochemical assay in the first reaction chamber to the current produced in the electrochemical assay in the second reaction chamber

wherein the complex mixture having an inhibitor of binding of the electrochemically labeled second member of the biological binding pair with the immobilized first member of the biological binding pair is identified by the production of a larger current in the first reaction chamber than is produced in the second reaction chamber. Specific interaction between the members of the biological binding pair is detected by a comparison of the electrical current produced in each reaction chamber when an electrical potential is applied between the electrodes in the chamber. The level and amount of current produced by specific binding of the first and second members of the biological binding pair in the reaction chamber is then compared with the level and amount of current produced in the chamber in the presence of a complex chemical mixture comprising an inhibitor of specific binding.

In an additional aspect of this embodiment of the invention, the method is used to isolate and identify an inhibitor of binding of the second member of the biological binding pair to the first member of the biological binding pair immobilized on the first electrode of the apparatus of the invention. In this embodiment, the method comprises the additional steps of:

d) chemically fractionating the complex mixture having an inhibitor of binding of the second member of the biological binding pair to the first member of the biological binding pair immobilized on the first electrode, to produce fractionated submixtures; and

e) performing steps (a) through (c) of the method on each of the fractionated submixtures to identify the submixtures that have an inhibitor of binding of the biological binding pair.

In this aspect, it will be recognized that steps (a) through (e) can be repeatedly performed on chemically fractionated submixtures to yield submixtures comprising increasingly purified preparations of the inhibitor. In preferred embodiments, the chemical fractionation includes chemical, biochemical, physical, and immunological methods for fractionation of chemical or biochemical species of inhibitor.

In preferred embodiments of each of the methods of the invention, the second member of the biological binding pair is an electrochemically labeled surrogate ligand for the first member of the biological binding pair, having an affinity of binding of from about 50 picomolar (pM) to about 0.5 mM, more preferably from about 1 nanomolar (nM) to about 100 micromolar (μM), and most preferably from about 10 nM to about 10 μM.

Specific preferred embodiments of the present invention will become evident from the following more detailed description of certain preferred embodiments and the claims.

### BRIEF DESCRIPTION OF THE DRAWINGS

FIG. 1 illustrates the arrangement of the components of the first electrode of the invention, comprising a conducting or semiconducting electrode, coated with an activated polymer or self-assembled monolayer to which a first member of a biological binding pair, a protein target, is immobilized thereto, which interacts with as electrochemically-labeled peptide that comprises the second member of the biological binding pair.

FIG. 2 illustrates the electrochemical analysis protocol using a GST-Src SH3 domain fusion protein and an electrochemically-labeled SH3 domain specific binding peptide.

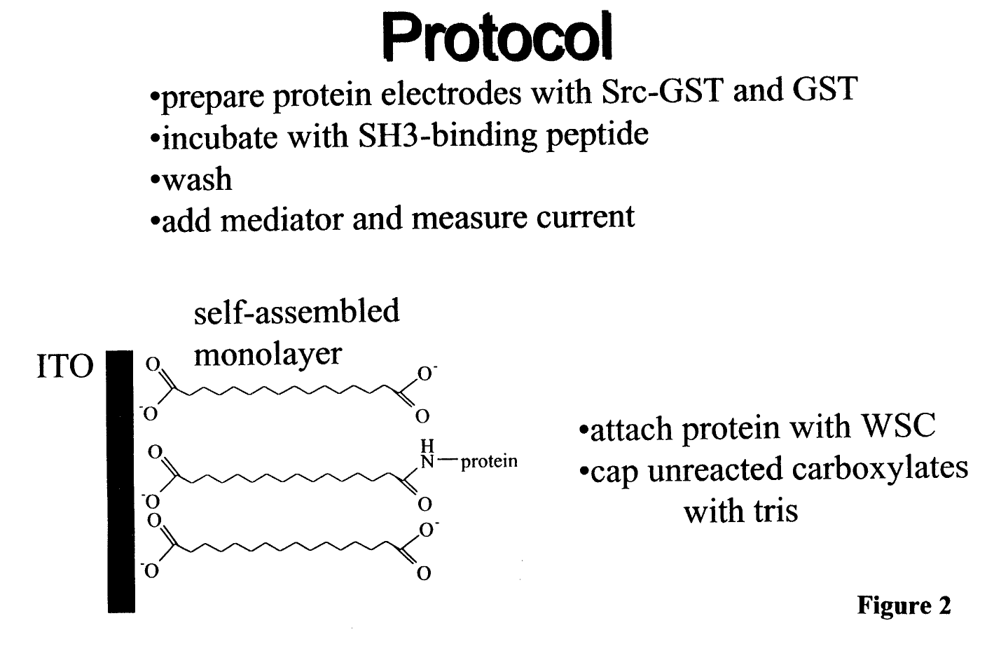

FIG. 3 shows the results of cyclic voltammetry using the protocol shown in FIG. 2.

FIG. 4 shows the results of integration and data manipulation of the cyclic voltammetry output of the experimental results shown in FIG. 3.

FIG. 5 is a graph showing the difference in integrated current output between the electrochemical reaction shown in FIG. 2 performed using an electrode having a GST-Src SH3 domain fusion protein immobilized thereto compared with the reaction performed using an electrode having GST alone immobilized thereto.

FIG. 6 shows the chemical reaction scheme for electrochemically labeling a peptide and the redox interaction of the labeled peptide with the electrochemical mediator.

FIG. 7 illustrates features of cyclic voltammetry methods.

FIG. 8 illustrates the current produced upon binding of src target protein and a surrogate ligand conjugated to horseradish peroxidase. The Figure also shows the current produced upon addition of a non-binding surrogate ligand. Hydrogen peroxide is added at 300 seconds followed by the surrogate ligand at 600 seconds.

FIG. 9 shows the currents measured upon binding of a surrogate ligand to a tyrosine RNA synthetase under the same conditions as in FIG. 8.

FIG. 10 shows the loss of current observed when a known inhibitor displaces the surrogate ligand from the tyrosine RNA synthetase.

FIG. 11 shows the current response upon concurrent addition of surrogate ligand and a known competitor the tyrosine RNA synthetase.

FIG. 12 shows the current response upon addition of surrogate ligand to tyrosine RNA synthetase which has been preincubated with inhibitor.

FIG. 13 shows the decrease in current response using a surrogate ligand in the presence of an increasing concentration of a tyrosine RNA synthetase competitive inhibitor.

FIG. 14 shows a graph of the relationship between the concentration of tyrosine RNA synthetase competitive inhibitor and the decrease in current response using the competitive inhibitor described in Example 11.

### DETAILED DESCRIPTION OF THE PREFERRED EMBODIMENTS

The present invention provides apparati and methods for detecting specific interactions, particularly including binding, between members of a biological binding pair. For the purposes of this invention, the term “biological binding pair” is intended to encompass any two biologically-derived or isolated molecules, or any chemical species that specifically interact therewith, that specifically bind with a chemical affinity measured by a dissociation constant of at least 50 mM. Specifically included in this definition of a biological binding pair are proteins that interact with other proteins, including fragments thereof; proteins and peptides; proteins and ligands; proteins and co-factors; proteins and allosteric or cooperative regulators; proteins and nucleic acids; proteins and carbohydrates; antigens and antibodies; lipids, including fatty acids, triglycerides and polar lipids that interact with proteins or peptides; receptors and ligands, particularly cytokines; virus-receptor pairs; enzymes and substrates; and enzymes and inhibitors. Also encompassed with this definition are any chemical compound or mixture that interacts with at least one member of a biological binding pair. The members of the biological binding pairs of the invention are intended to encompass molecules that are naturally-occurring, synthetic, or prepared by recombinant genetic means or biochemical isolation and extraction means. Synthetic embodiments of a member of a biological binding pair will be understood to typically share structural similarity with at least a portion of any naturally-occurring analogue which they resemble or are constructed to resemble or mimic. These definitions are non-exclusive and non-limiting, and are intended to encompass any two biological or chemical species capable of specifically interacting with the defined chemical affinity.

The apparatus of the invention comprises a first, conductive or semiconductive electrode coated with a porous, hydrophilic, polymeric material. Non-limiting examples of materials useful for preparing the conductive or semiconductive electrodes of the invention include metallically-impregnated glass, such as tin-doped indium oxide or fluorine-doped tin oxide glass, gold, carbon or platinum. Examples of materials useful as coatings for the first electrode of the invention include agar, agarose, dextrans and modified dextrans, acrylamide, pyrroles and pyrrole-carboxylates, polystyrene, nylon, nitrocellulose, mylar, Nafion, polyethylene, polypropylene, polypyrroles, polythiophene, and polyaniline. The coating of the first electrodes of the invention are prepared using methods dependent on the chemical nature of the coating species and the conductive or semiconductive electrode material. For example, electrode surfaces can be coated by electropolymerization using pyrroles, or by spin-casting, evaporation or in situ polymerization using soluble supports such as polystyrene, mylar or Nafion. These coatings are optimized for tolerance to unbound impurities, for example, by regulating their thickness. Members of a biological binding pair such as proteins are then attached to the electrode using a variety of chemical conjugation techniques that are dependent on the nature of electrode coating material. For example, carbodiimide crosslinking is useful when the electrodes contain oxidized mylar on metal oxide, carbon or gold, oxidized polystyrene on carbon or gold, alkanethiol-carboxylate self-assembled monolayers (SAMS) on gold, carboxylate SAMS on metal oxides, or electropolymerized carboxylate-containing monomers. Alternatively, avidin or streptavidin can be attached to the electrode by any of the above means or by passive adsorption to the polymeric coating, and a biotin-conjugated target protein is then bound via its interaction with avidin or streptavidin. Additionally, a poly-histidine-tagged target may be bound to an electrode that has a coating that can bind divalent nickel ions (Ni²⁺). These methods are chosen and optimized for protein and temperature stability, solvent accessibility of bound ligand and ligand-binding efficiency.

The apparatus of the invention also provides a second member of a biological binding pair, wherein said second member is electrochemically labeled. Electrochemical labels are defined as chemical species, typically cationic species comprising cations including ruthenium, osmium or cobalt, that are capable of participating in a reduction/oxidation (redox) reaction with the electrochemical mediator and the first electrode of the apparatus when an electrical potential is applied between the electrodes in the reaction chamber of the apparatus. For second members of a biological binding pair comprising a peptide, inorganic complexes such as Ru^{2+/3+}-amine complexes, ferrocenes, and osmium- or cobalt-polypyridyl complexes are attached to the peptide via histidine or cysteine residues or at the amino terminus. Redox-active organic molecules, such as paraquat derivatives and quinones, are attached to peptides by conjugating the redox-active organic moiety via lysine or cysteine residues or at the amino terminus.

Such redox-active organic and inorganic molecules are also used as electrochemical mediators in the electrolyte solution of the reaction chamber of the apparatus of the invention, whereby the mediator is chosen for electrochemical compatibility with the electrochemical label used. The choice of the combination of the electrochemical label and mediator is optimized for current sensitivity, specificity of label and capacity to diffuse within the polymer matrix of the semiconductive electrode coating.

In the practice of the invention, preferred compounds comprising the second member of the biological binding pair are “surrogate” ligands to the first member of the specific binding pair. For the purposes of this invention, the term “surrogate ligand” is intended to define a set of biologically-active compounds that specifically bind to any defined target comprising a first member of a biological binding pair. Although this definition is intended to encompass a variety of ligands of a target, particularly a target protein, comprising the first member of a biological binding pair, including a natural ligand, the surrogate ligands of the invention preferably comprise those ligands that specifically bind to the target protein with a chemical affinity measured by a dissociation constant (K_{d}) of from about 50 picomolar (pM) to about 0.5 mM, more preferably from about 1 nanomolar (nM) to about 100 micromolar (μM), and most preferably from about 10 nM to about 10 μM. Such surrogate ligands are preferred because they bind with sufficient affinity that the concentration of the electrochemical label at the surface of the first electrode of the apparatus of the invention is sufficient to produce an experimentally-detectable current, while at the same time the binding affinity is weak enough to be displaced by competitors and inhibitors at concentrations of these compounds that are economical and can be experimentally achieved. Surrogate ligands therefore provide both the required degree of specificity and the required degree of easy dissociability to enable the methods and apparatus of the invention to detect binding inhibition by competitor species. Some of the targets for which binding peptides have been identified are listed in Table I.

In the practice of the methods of the invention, second members of the biological binding pair that are electrochemically-labeled surrogate ligands include, but are not limited to, peptides, nucleic acids, carbohydrates, and small molecules. In embodiments of the methods of the invention using peptides as second members of the biological binding pair, the peptides are preferably obtained from phage-displayed combinatorial peptide libraries (see co-owned and co-pending U.S. patent application Ser. No. 08/740,671, filed Oct. 21, 1996, incorporated by reference herein) as well as other means, such as synthetic peptides prepared on pins or beads. Such peptides that contain only naturally-occurring amino acids must be electrochemically-labeled, because they lack sufficient redox potential under the voltage conditions tolerated by the first member of the biological binding pair that is the electrode-immobilized target. Peptides and proteins comprising the electrochemical probes and targets of the apparati and methods of this invention can be prepared by synthetic methods, including solid phase peptide synthesis, biochemical isolation and modification techniques including partial proteolysis, and by recombinant genetic methods understood by those with skill in the art (see Sambrook et al., 1990, Molecular Cloning, 2d ed, Cold Spring Harbor Laboratory Press, N.Y.).

An example of a useful electrochemical labeling method is addition of a ruthenium group (RU(NH₃)₅(OH₂)₂²⁺) to histidine residues within the peptide sequence. Alternatively, electrochemical labels can be added to the amino- or carboxyl termini post-synthetically, or to the reactive side chain thiol groups of a cysteine residue, the hydroxyl group of a serine or threonine residue (or on a carbohydrate moiety), or the amino group of a lysine residue of the peptide. In addition, Fmoc derivatives of “unnatural” amino acids (such as D-amino acids or amino acid analogues such as ε-aminocaproic acid) that can be incorporated during peptide synthesis can be used.

In addition, a variety of non-peptide surrogate ligands can be adapted to this electrode system. For example, nucleic acids (i.e., RNA- and DNA-species, including poly- and oligonucleotides) that specifically bind to a target molecule can be obtained from combinatorial nucleic acid libraries; these molecules have been termed “aptamers” (as disclosed in Gold et al., 1995, Ann. Rev. Biochem. 64: 763). Such aptamers can be electrochemically-labeled with a labeling group at either the 3′ or 5′ termini, or modified nucleotide triphosphate that binds an electrochemical labeling group can be incorporated into oligonucleotides by non-discriminating RNA or DNA polymerases during the in vitro generation of the aptamer. Finally, certain small molecules can be electrochemically-labeled in a way that does not destroy their binding activity. For example, cyclic AMP (cAMP) can be electrochemically-labeled without diminishing binding to protein kinase A, thereby providing a biological binding pair for electrochemical analysis of compounds that affect cAMP binding to protein kinase A.

Electrolyte solutions useful in the apparatus of the invention include any electrolyte solution

TABLE I
Targets for which binding peptides have been
identified from combinatorial libraries

| Targets | References |
|---|---|
| Streptavidin | 1, 2, 3 |
| HLA-DR | 4, 5 |
| concanavalin A | 6, 7 |
| calmodulin | 8, 9 |
| S100 | 10 |
| p53 | 11 |
| SH3 domains | 12-18 |
| Urokinase receptor | 19 |
| bFGF-R | |
| integrin IIb/IIIa/avB1 | 20-23 |
| Hsc70 | 24 |
| tissue factorVIIa | |
| atrial naturiuretic peptide A receptor | |
| fibronectin | 25 |
| E-selectin | 26 |
| CD1-B2M complex | 27 |
| tissue-type plasminogen activator | 28 |
| core antigen of Hepatitis B virus | 29 |
| HIV-1 nucleocapsid protein NCp7 | 30 |
| erythropoietin receptor | 31 |
| trypsin | 32 |
| chymotrypsin | 33 |
| interleukin-1 receptor | 34 |
| References: 1. Devlin et al., 1990 Science 249: 404; 2. Lam et al., 1991, Nature 354: 82; 3. Fowlkes, 1993, Gene 128: 59; 4. Hammer et al., 1992, J. Exp. Med. 176: 1007; 5. Hammer et al., 1993, Cell 74: 197; 6. Scott et al., 1992, Proc. Natl. Acad. Sci. USA 89: 5398; 7. Oldenburg et al., 1992, Proc. Natl. Acad. Sci. USA 89: 5393; 8. Dedman et al., 1993, J. Biol. Chem. 268: 23025; 9. Adey et al., 1996 Gene | |
| # 169: 133; 10. Ivanenkov et al., 1995, J. Biol. Chem. 270: 14651; 11. Daniels et al., 1994, J. Mol. Biol. 243: 639; 12. Sparks et al., 1996, Proc. Natl. Acad. Sci. USA 93: 1540; 13. Sparks et al., 1994, J. Biol. Chem. 269: 23853; 14. Cheadle et al., 1994, J. Biol. Chem. 269: 24034; 15. Rickles et al., 1994, EMBO J. 13: 5598; 16. Rickles et al., 1995, Proc. Natl. Acad. Sci. USA 92: 10909; 17. Chen et al., 1993, J. | |
| # Amer. Chem. Soc. 115: 12591; 18. Yu et al., 1994, Cell 76: 933; 19. Goodson et al., 1994, Proc. Natl. Acad. Sci USA 91: 7129; 20. O'Neill et al., 1992, Proteins 14: 509; 21. Fong et al., 1994, Drug Dev. Res. 33: 64; 22. Kolvunen et al., 1993, J. Biol. Chem. 268: 20205; 23. Kolvunen et al., 1993, J. Cell Biol. 124: 373; 24. Takenaka et al., 1995, J. Biol. Chem. 19839; 25. J. Cell Biol. 130: 1189; 26. Martens et al., | |
| # et al., 1995, J. Biol. Chem. 270: 21129; 27. Science 269: 223; 28. Proc. Natl. Acad. Sci. USA 92: 7627; 29. Dyson et al., 1995, Proc. Natl. Acad. Sci. USA 92: 2194; 30. FEBS Lett. 361: 85; 31. Wrighton et al., 1996, Science 273: 458; 32. Fang et al., 1996, Biochem. Biophys. Res. Commun. 220: 53; 33. J. Chromatography 711: 119; 34. Yanofsky et al., 1996, Proc. Natl. Acad. Sci. USA 93: 7381. | |

at physiologically-relevant ionic strength (equivalent to about 0.15M NaCl) and neutral pH. Nonlimiting examples of electrolyte solutions useful with the apparatus of the invention include phosphate buffered saline (PBS), HEPES buffered solutions, and sodium bicarbonate buffered solutions.

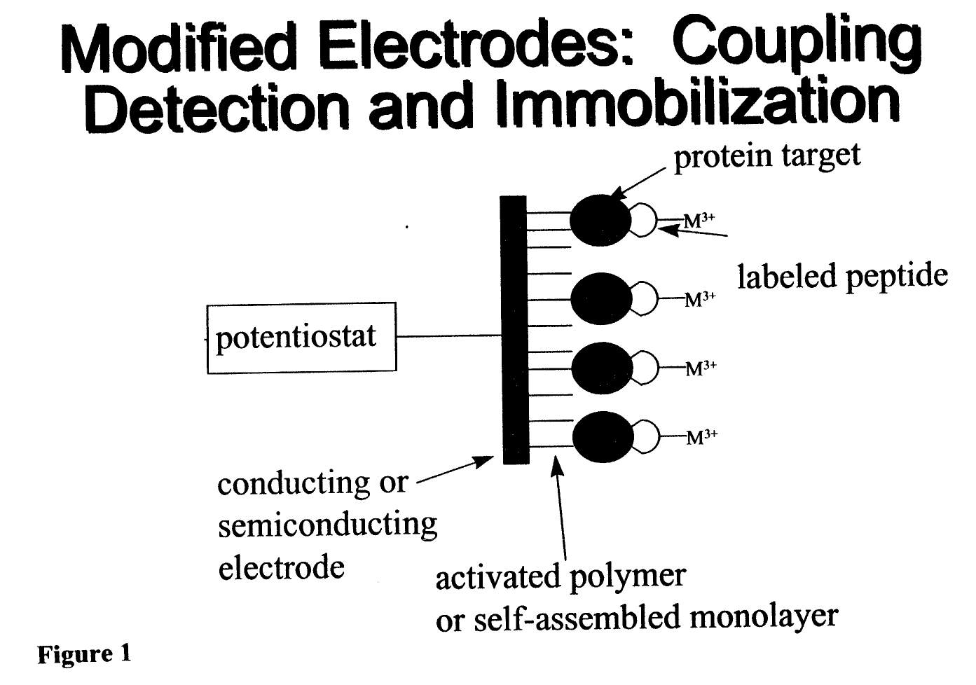

In the practice of the methods of the invention, when a current is applied to the electrode, electrons are transferred from the electrochemical label to the mediator and then to the electrode by a series of redox reactions between the components of the reaction. However, redox electron transfer is only maximal (and a detectable current is only produced) when the electrochemical label is highly concentrated at the electrode surface. This will only occur when the electrochemically-labeled second member of the biological binding pair is bound to the first member of the biological binding pair immobilized on the electrode as the target protein. This schema is illustrated in FIG. 1.

In one application of the methods of the invention are provided high-throughput screens of natural product and combinatorial chemical libraries for antagonists of protein-protein interactions. Such low molecular weight chemical antagonists of specific protein-protein interactions are of value to the pharmaceutical industry as potential drug leads for developing therapeutic agents. In the practice of these methods of the invention, a target protein comprising a first member of a biological binding pair is immobilized on an electrode surface. This first electrode is placed in a reaction chamber of the apparatus of the invention, preferably a microtiter plate well, said reaction chamber containing an electrochemically-labeled surrogate ligand and a compound or mixture of compounds to be tested for the ability to inhibit binding of the surrogate ligand to the target protein. (It will be understood that fragments of biologically-active proteins retaining the specific binding properties thereof are also encompassed within the scope of the target proteins of the electrodes of the invention.) For example, each of the reaction chambers or microtitre sample wells in a representative experiment can contain discrete combinatorial compounds or purified natural products (such as polyketides or fermentation broth components). After incubating the compounds in the presence of the electrode, potentiometric analysis of the current produced in the reaction chamber is performed; preferably, this analysis is cyclic voltammetry. The results of these analyses are compared for wells containing the electrochemically-labeled surrogate ligand in the presence and absence of the compound or mixture of compounds to be tested. Compounds that inhibit the binding of the electrochemically-labeled surrogate ligand to the immobilized target protein on the electrode surface yield a reduced amount of current compared with compounds that do not bind to the target, which show no effect on surrogate ligand binding. In the practice of this invention, appropriate controls for detecting reduction in observed current due to target protein denaturation are included in each determination. These controls include testing the experimental electrode in a reaction chamber freshly prepared with a solution comprising an electrochemical mediator and electrochemically-labeled surrogate ligand in the absence of the compounds to be tested, and by testing these compounds using electrodes coated with unrelated target proteins. Optimally, the methods of the invention are practiced on a 96-well microtitre plate whereby 96 electrodes are configured to be utilized simultaneously. In other embodiments, multiple electrodes comprising different target proteins immobilized thereto are in electrical contact with each well and are used to evaluate a single compound for inhibitory capacity against binding of an array of different targets comprising the first member of a biological binding pair with a variety of different electrochemically-labeled surrogate ligands comprising the second member of a biological binding pair.

Alternatively, the competition binding assays are performed to detect compounds that affect specific binding between the target protein and the electrochemically-labeled surrogate ligand by causing a conformational change in the target protein. In these embodiments of the methods of the invention, the electrode is first incubated with the electrochemically-labeled surrogate ligand, washed and then placed in a reaction chamber containing the compound or compounds to be tested. Compounds that bind to an available site on the target and induce a conformational or allosteric change in the target cause release of the electrochemically-labeled surrogate ligand, and are detected by the production of a decrease in the observed current in the reaction chamber as detected, for example, by cyclic voltammetric analysis. As above, appropriate control reactions are performed to detect loss of surrogate ligand binding due to target protein denaturation.

The invention also provides methods for measuring the binding affinity of interaction between members of a biological binding pair, such as protein-peptide and protein-protein interactions. These measurements are useful for determining the dissociation constant (K_{d}) of the interaction between the components of the biological binding pair. These methods provide an alternative to existing methods for measuring binding affinities and dissociation constants, such as surface plasmon resonance instruments (e.g., BIAcore®, Pharmacia). The methods of the present invention are advantageous with compared with such previously-disclosed technologies because the present methods are more rapid, less costly and require less biological material. In addition, the methods of the present invention can be practiced using electroprobes and electrochemical ligands having molecular weights of 300 daltons or more. In contrast, the methods known in the prior art require ligands that are at least about 5 kilodaltons in size, since the signal strength using prior art methods is proportional to the size of the binding ligand. This limitation prevents analysis of binding interaction properties of molecules having a molecular weights less than the cutoff threshold, 5 kD. This limitation is important, since small molecular weight compounds form a large percentage of potential drug lead compounds. In addition, assay conditions using the methods and apparati of this invention are more permissible than the assay conditions required using the methods of the prior art, including but not limited to conditions of probe concentration, salt concentration and assay performance in the presence of organic solvents.

The invention also provides methods and apparati for determining the binding affinity and chemical “strength” of the interactions between members of a biological binding pair. Knowing the strength of the interaction between two members of a biological binding pair is important for determining whether the interaction has potential as a good target for drug discovery. The ability to detect these interactions with a rapid, inexpensive and convenient assay can greatly accelerate both target validation and screening. The methods of the present invention provide the ability to screen any two members of a biological binding pair for the capacity to specifically bind or otherwise specifically interact. The invention also provides methods for mapping region(s) of interaction between the members of the pair, using various truncated or altered forms of either or both members of the binding pair. For protein-protein interactions, there are several currently of interest in drug discovery, that are listed in Table II.

In yet another embodiment of the methods of the invention are provided methods for detecting specific binding and other interactions between members of biological binding pairs in complex mixtures of chemical and biochemical molecules. In one embodiment, protein:protein interaction methods are provided. Such interactions are difficult to detect or characterize using existing technology. Using the methods of the present invention, an electrode coated with a particular target protein is incubated with an electrolyte solution containing an electrochemically-labeled surrogate ligand and a cell extract comprising a protein(s) that specifically interacts with the target protein on the electrode. As described with other embodiments of competition binding experiments using the methods of the invention, binding of the interacting protein instead of the electrochemically-labeled surrogate ligand results in a decreased amount of current produced during electrochemical analysis, e.g., cyclic voltammetry.

This inventive method for detecting protein-protein interactions is advantageous compared with currently-available methods, as illustrated by a comparison with current methods for assaying column fractions during protein purification. The currently-available techniques include enzymatic assays of chromatographic column fractions that generate a radioactive product and that are only applicable to proteins having known enzymatic activity. For proteins with unknown enzymatic activity, ELISA assays, band shift assays using a radiolabeled target, or co-immunoprecipitations (that require antibodies to a radiolabeled target) are used. Each of these methods is time-consuming and tedious, and frequently require the use of radiochemical detection methods that are disadvantageous in terms of safety and regulatory concerns.

In contrast, the methods of the current invention are rapid, specific, and inexpensive. An additional advantage of the electrochemical screening methods of the present invention is that such screening methods are able to detect weak protein-protein interactions that cannot be detected by existing techniques. The methods of the present invention are also applicable to a variety of alternative embodiments of protein purification techniques, including analysis of chromatographic fractions, tissue distribution surveys for the presence of the target binding protein in tissue samples from tumors, and for cell-cycle specific interactions, for example, by using extracts from synchronized cells.

The methods and apparati of the invention are advantageous over the analytical techniques

TABLE II
Interactions of Interest

| Molecule 1 | Molecule 2 |
|---|---|
| fibronectin | integrin |
| antigen | antibody |
| calmodulin | ˜20 effector molecules |
| tubulin | microtubule associated proteins |
| actin | actin binding proteins, Dnase I |
| p53 | MDM2 |
| cdk | cyclin, p21 |
| ras | raf |
| fos | jun |
| TBP | RNA polymerases |
| Sos | Grb2 |
| p53 | p53BP2 |
| K-channel | src |
| various proteins | WW domain containing proteins |
| ptyr proteins | SH2 domains, PTB domains, |
| | phosphatases |
| UGI | UDG |
| regulatory subunit PKA | catalytic subunit PKA |
| enhancer elements | enhancer binding proteins |
| DNA | transcription factors |
| RNA | RNA binding proteins |
| concanavalin A | lectins |
| lipids | lipoproteins |
| fatty acids (FA) | FA binding proteins |
| steroids | steroid hormone receptors |
| cytomegalovirus DNA polymerase | polymerase accessory factor |
| BPTI | trypsin |
| Rb | E2F, E1A, SV40 T antigen |

References: Iwabuchi et al., 1994, Proc. Natl. Acad. Sci. USA 91: 6098; Holmes et al., 1996, Science 274: 2089; Rozakis-Adcock et al., 1993, Nature 363: 83; Phizicky et al., 1995, Microbiol. Rev. 59: 94; Chan et al., 1996, EMBO J. 15: 1045; Chen et al., 1995, Proc. Natl. Acad. Sci. USA 92: 7819; Sudol et al., 1995, FEBS Lett. 369: 67.

and equipment known in the art for the following reasons. First, the sensitivity of the methods of the invention permit detection of specific binding interactions between the members of a biological binding pair over 4-5 orders of magnitude of concentration (i.e., 10,000- to 100,000-fold). This invention provides detection methods having the sensitivity of radiochemical detection methods without the health, safety and regulatory concerns that accompany radiochemically-based methods. The invention also affords detection of biological binding interactions with high sensitivity over a wide range of binding affinities. Second, the assays are rapid, inexpensive and are performed in vitro. Third, the reagents used in the practice of the invention (i.e., the electrodes and electrochemically-labeled surrogate ligands) are stable and have a relatively long shelf-life compared with, for example, radiochemical reagents. Fourth, structure-activity relationships can be determined quantitatively, based on the determination of changes in drug binding kinetics observed using cyclic voltammetry, for example. Fifth, the analyses can be multiplexed, that is, each reaction can be performed in a reaction chamber comprising more than one immobilized target protein-comprising electrode, so that one or a mixture of potential drug lead compounds can be analyzed for binding to a variety of potential targets. Sixth, the methods and apparati of the invention are amenable to automation, including but not limited to the use of multiwell (such as 96-well microtitre) assay plates and robotic control of electrodes and electrochemical components of the reaction chambers thereof. Seventh, the sensitivity of the electrochemical assays of the invention permit detection of small amounts (about 50,000 electrochemical labels bound to the target) of either surrogate ligand, inhibitory compounds, or both, thereby increasing the efficiency of performing assays such as drug screenings. Eighth, these increases in efficiency result in higher throughput screening, addressing a major obstacle to drug development. Ninth, the invention provides methods for determining dissociation constants for biological binding pair interactions that are more rapid, less expensive and require less sample than known methods (including, for example, equilibrium dialysis, analytical ultracentrifugation, analytical microcalorimetry and BIAcore®-analysis). Tenth, the assays provided by the present invention can be performed in the absence of any information on the identity of the binding partner for any target protein or surrogate ligand. This advantage eliminates the requirement that the biological activity of a target protein be known before the protein can be characterized. Eleventh, the assays of the invention are flexible, and allow analysis of binding or competition binding for any biological binding pair. Moreover, either of the binding pairs can be electrochemically-labeled, and under appropriate assay conditions, both members of the biological binding pair can be in the electrolyte solution in the reaction chamber.

The apparatus of the invention also provides a hydrophilic polymer modified electrode containing the first member of a biological binding pair, preferably a protein and most preferably a receptor or fragment thereof, and an immobilized electrochemical mediator. Said first members of a biological binding pair, such as proteins, and electrochemical mediators are chemically linked to the polymeric support either directly through covalent bond formation between reactive groups or through mutually reactive chemical linkers. For example, the side chains of several amino acids contain nucleophilic heteroatoms that can undergo addition to epoxide functionalities in polyethylene glycol diglycidylether. Alternatively, the nucleophiles present in a polymer such as polylysine can be linked to protein via bifunctional activated electrophiles such as dicyclohexylcarbodiimide-, N-hydroxysuccinimide-, or hydroxybenzotriazole-activated dicarboxylates. Techniques for coupling electrochemical mediators include coordination of a transition-metal complex to nucleophilic atoms on the polymer, incorporation of a reactive group into an organic mediator or metal-complex ligand, or incorporation of transition-metal-binding sites along the polymer backbone. For example, coordination of polyvinylimidazole to bisbipyridinechloroosmium(II) yields a very stable polymer in which Os(II) and Os(III) interconvert at modest applied potentials. Chemical modifications of bipyridine ligands have resulted in metal complexes containing activated carboxylate moieties for coupling to nucleophiles and other functional groups that allow direct incorporation of complexes in the context of automated biopolymer synthesis.

In these embodiments of the apparatus of the invention are also comprised a second member of a biological binding pair, preferably a peptide or nucleic-acid surrogate ligand as defined herein, coupled to an electrochemical catalyst comprising an electrochemically activated catalytic species. Examples of such electrochemical catalysts are enzymes such as glucose oxidase and horseradish peroxidase, which effect the oxidation or reduction of their substrates and are electrochemically reactivated at potentials that are insufficient to effect direct electrochemistry of the substrate. Such enzymes are understood in the art to achieve catalysis by lowering an electrochemical barrier in the redox chemistry of the substrate, so that judicious choice of electrode potential allows selective electrochemical detection of the enzyme-catalyzed reaction in the vicinity of the electrode. Also, several synthetic transition-metal complexes such as those of oxoruthenium(IV), oxoosmium(IV), oxomolybdenum(IV), dioxomolybdenum(VI) and dioxorhenium(VI) are capable of oxidizing or reducing a variety of organic functional groups in a substrate at potentials at which direct electrochemistry is impossible. (For examples, see Stultz et al., 1995, J. Am Chem. Soc. 117: 2520; Cheng et al., 1995, J Am. Chem. Soc. 117: 2970, Neyhart et al., 1993, J. Am Chem. Soc. 115: 4423; Schultz et al., 1993, J. Am Chem. Soc. 115: 4244; Thorp et al., 1989, Inorg. Chem. 28: 889).

In the use of this embodiment of the invention, binding of the second member of the biological binding pair to the first member of the biological binding pair concentrates the electrocatalyst in proximity to the electrode and mediators immobilized thereon. Redox reactions between the electrocatalyst, the substrate and the electrochemical mediator results in current flow at the electrode, due to transfer of redox equivalents to the substrate. When a sufficient potential is applied to the electrode, the immobilized mediators are either completely oxidized or completely reduced. For example, using a surrogate ligand linked to horseradish peroxidase, binding of the surrogate ligand at the electrode surface with the first member of a biological binding pair brings the horse radish peroxidase enzyme in sufficient proximity to the reduced electrochemical mediators on the electrode to reduce the enzyme itself. This reduced form of the enzyme is the active form, which can therefore act catalytically to transfer electrons to hydrogen peroxide in the solution, producing oxygen and water. The enzyme is constitutively reduced by the electrochemical mediators in the polymer comprising the electrode after each catalytic cycle and, as the entire process is repeated, the binding of the surrogate ligand is detected and quantitated as current flowing through the electrode to the solution. Thus, under the appropriate conditions of substrate concentration and applied potential at the electrode, the amount of current produced is proportional to the amount and extent of binding of the members of the biological binding pair at the electrode surface.

One application of the methods of the invention provided herein is a means for measuring the binding kinetics of a biological binding pair. In the absence of the surrogate ligand-enzyme conjugate (or in the presence of enzyme linked to a non-binding species), very little current is transferred from the polymer-modified electrode to the enzyme substrate. Upon addition of a conjugate between an electrochemical catalyst and a surrogate ligand with binding specificity for the immobilized first member of the biological binding pair, and in the presence of an sufficient concentration of a substrate for the electrochemical catalyst, current increases dramatically and eventually achieves a stable, steady-state value. Since the electrode potential is chosen to effect instantaneous reactivation of the enzyme and the substrate is present in the solution in large excess, current increases as a reflection of increasing numbers of bound surrogate ligand conjugates at the electrode surface until all possible binding sites are occupied. The current increases with a typical rate constant that is the rate constant of the binding reaction; the invention thereby provides an efficient means for measuring said rate constant. Conversely, the dissociation rate constant can be measured by immersing a conjugate-saturated electrode in a solution free of conjugate, and measuring the rate of decrease in produced current. A knowledge of these binding rates and strengths is vital to the understanding of interactions between biomolecules, including but not limited to protein-protein, protein-drug and protein-nucleic drug binding phenomena.

In another application of the invention, binding or lack of binding of the conjugate is used to determine the occupancy of the available binding sites by an electrochemically inactive species. Typically, this species will be a single drug candidate from a large library of either natural products or combinatorially synthesized molecules. Binding of the drug candidate can be ascertained by at least three related techniques. In a first embodiment, the electrode can be preincubated with the drug candidate to allow all possible binding interactions between the candidate drug and the electrode-immobilized first member of the biological binding pair to occur prior to adding the surrogate ligand conjugate. The decrease in current upon addition of the conjugate, when compared with current produced in the absence of the drug candidate, is a measure of the extent of occupancy of the available binding sites of the electrode-immobilized first member of the biological binding pair by the drug. In a second embodiment, a drug candidate is added concurrently with a surrogate ligand conjugate at different concentrations, and the effect of the presence of the drug candidate on the produced current used to determine the inhibition constant of the drug for surrogate ligand binding. In a third embodiment, the electrode can be saturated with conjugate prior to the addition of the drug, whereby loss of observable current indicates the capacity of the drug candidate to displace surrogate ligand binding. Those of ordinary skill will recognize the utility of these methods for characterizing the binding and inhibitory properties of drug candidates for any biological binding pair of interest.

Additional features of the invention are more fully illustrated in the following Examples. These Examples illustrate certain aspects of the above-described method and advantageous results. These Examples are shown by way of illustration and not by way of limitation.

### EXAMPLE 1

### Preparation of Electrochemically Labeled Peptides

Electrochemically labeled peptides were prepared using art-recognized techniques (see Yocom et al., 1982, Proc. Natl. Acad. Sci. USA 79: 7052-7055; Nocera et al., 1984, J. Amer. Chem. Soc. 106:5145-5150). In one example, the derivatized peptide NPDF-1, having the amino acid sequence:

Gly-His-Gly-Ser-Gly-Arg-Ala-Leu-Pro-Pro-Leu-Pro-Arg-Tyr-NH₂ (SEQ ID NO.: 1)

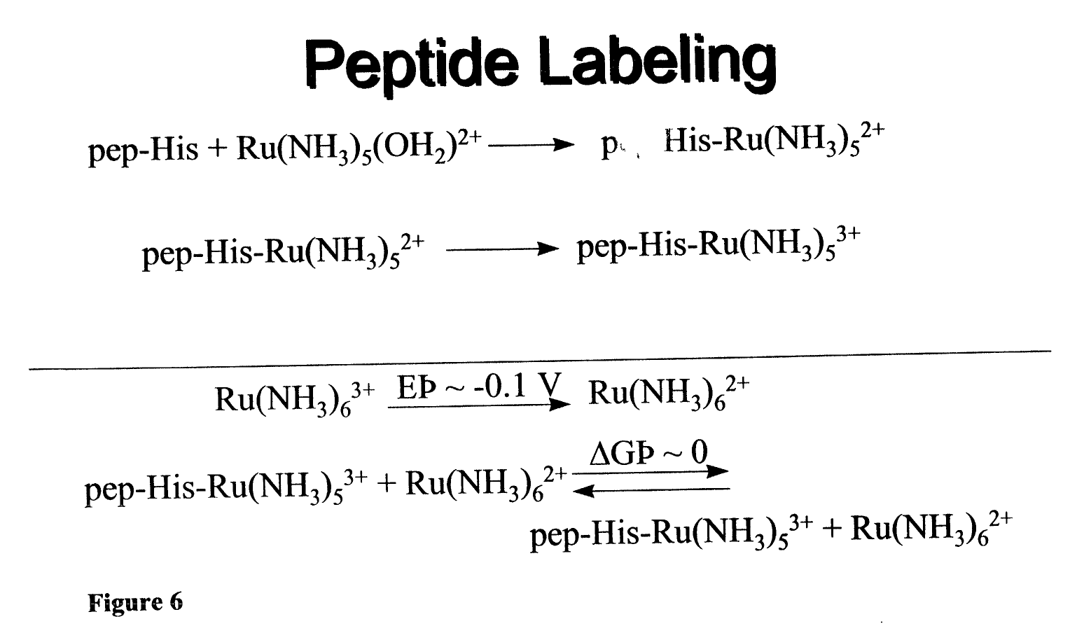

is labeled as follows. The electrochemical label Ru(NH₃)₅(H₂O)²⁺ was generated by the reduction of {Ru(NH₃)₅Cl}Cl₂ over zinc/mercury amalgam, using conventional techniques (see Ford et al., 1968, J. Amer. Chem. Soc. 90: 1187-1194; Vogt et al., 1965, Inorg. Chem. 4: 1157-1163). Peptide (about 5 mg) at a concentration of about 0.2 mM was reacted with a fifty-fold molar excess (˜10 mM) Ru(NH₃)₅(H₂O)²⁻ under argon atmosphere at room temperature in 50 mM sodium phosphate buffer (pH 7.0) for 48 hr (FIG. 6). The reaction was terminated by applying the solution to a Sephadex G-25 column (Pharmacia, Upsala, Sweden), equilibrated with 50 mM buffer. Fractions from this column containing peptide were pooled, oxidized and concentrated. The components of the oxidized mixture were then separated by cation exchange chromatography using a Whatman CM-cellulose (CM52) chromatography column that had been equilibrated with 50 mM phosphate buffer (pH 7.0) at 4° C. Electrochemically-labeled peptide-containing fractions were collected and maintained at 4° C. until use.

### EXAMPLE 2

### Preparation of Protein-immobilized Electrodes

Tin-doped indium oxide electrodes were purchased (Delta Technologies) as 2 cm² square glass slides and prepared for use as follows. The electrode was cleaned by sonication treatment in a laboratory sonicator by sequential treatment in Alkonox, neat isopropyl alcohol, distilled and deionized water (three times), and finally the desired buffer; each sonication treatment having been performed for 10 min. The cleaned electrodes were then immersed in a 5 mM solution of 1,12-dodecadicarboxylic acid and incubated at room temperature for 48-72 hours, followed by rinsing the electrodes with hexane (Analytical grade).

Protein crosslinking to the prepared electrode was then performed as follows. A 50 μL aliquot of a 5 mM solution of 1-(3-dimethylaminopropyl)-3-ethylcarbodiimide was placed on one side of the electrode and dried at room temperature. 20 mL of a 4 mg/mL solution of a fusion protein in phosphate buffered saline (PBS)/0.1% Tween 20 was placed on the surface of the dried electrode previously treated with carbodiimide and incubated at 4° C. overnight to allow crosslinking to proceed. After this incubation, electrodes were washed once with a solution of 100 mM Tris-HCl (pH8.0)/100 mM NaCl for 5 min. and kept in PBS at 4° C. until used.

### EXAMPLE 3

### Cyclic Voltammetry

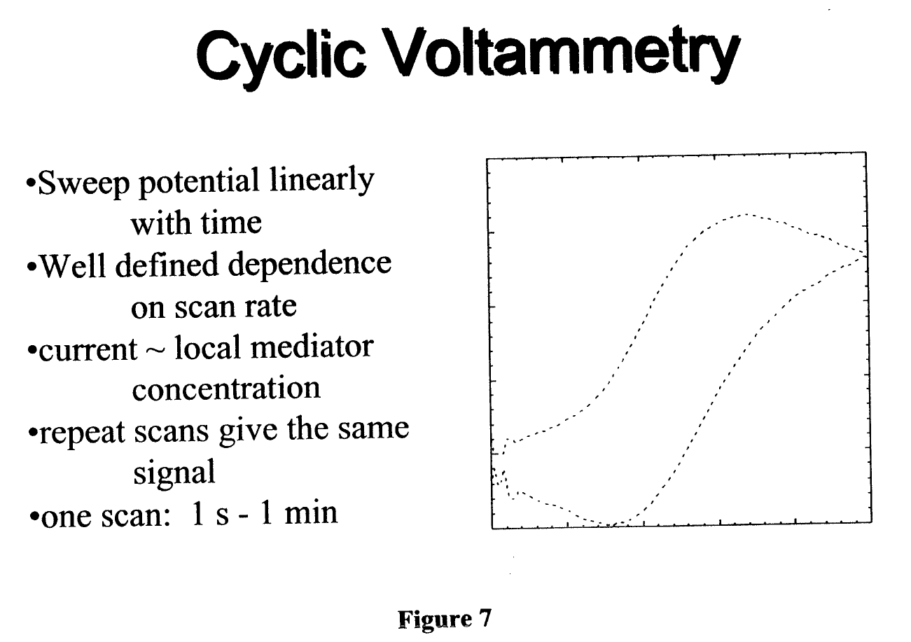

Cyclic voltammograms were collected using an EG&G PAR 273A potentiostat/galvanostat with a single compartment voltammetric cell equipped with a modified tin-doped indium oxide (ITO) working electrode (area=0.32 cm²), platinum (Pt)-wire counter electrode and silver/silver chloride (Ag/AgCl) reference electrode (see Johnston et al, 1994, Inorg. Chem. 33: 6388-6390). An example of such a voltammogram is shown in FIG. 7 and was generated under the following conditions. A sample containing 50 mM Ru(NH₃)₆³⁺ and 20 mM Ru(NH₃)₅³⁺-labeled peptide dissolved in buffered aqueous solutions containing 50 mM sodium phosphate buffer (pH 6.8)/700 mM NaCl (having a total sodium ion concentration of 780 mM) was scanned at a rate of 15 mV/s. After scans with the electrochemically labeled peptide, the electrode was rinsed with the same 50 mM sodium phosphate buffer solution, and then with 50 mM Ru(NH₃)₆³⁺ or an unlabeled peptide solution containing 50 mM Ru(NH₃)₆³⁺. Cyclic voltammograms were obtained with 50 mM Ru(NH₃)₆³⁺ after washing. Scans of Ru(NH₃)₅³⁺-labeled peptide in the absence of electrochemical mediator showed no appreciable reduction current to −0.2 V vs. Ag/AgCl. A freshly prepared electrode was used for each experiment and a background scan of buffer alone was collected for each electrode and subtracted from subsequent scans.

### EXAMPLE 4

### Electrochemical Assay for Detecting Specific Binding

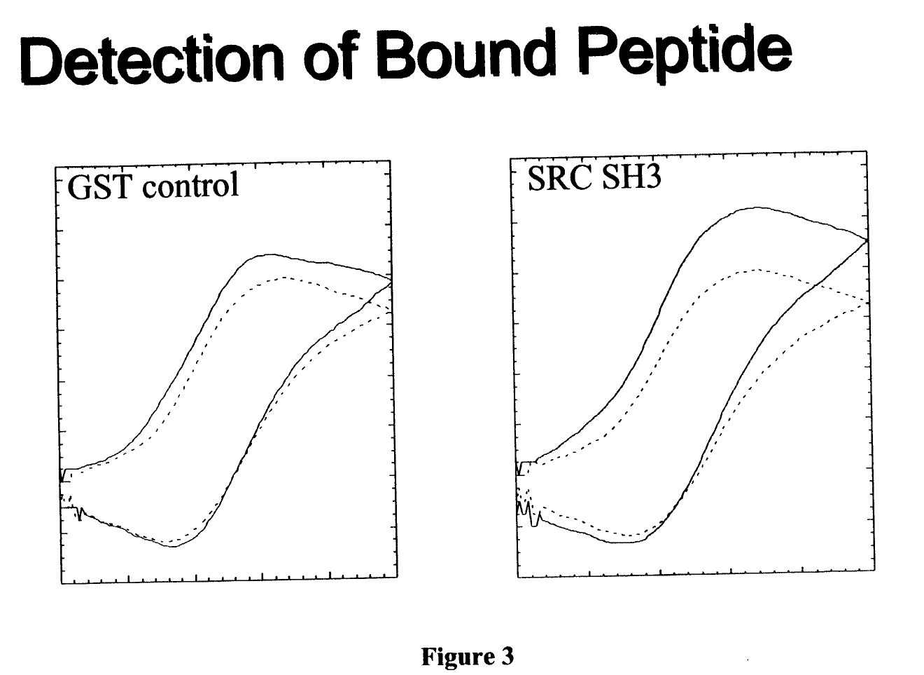

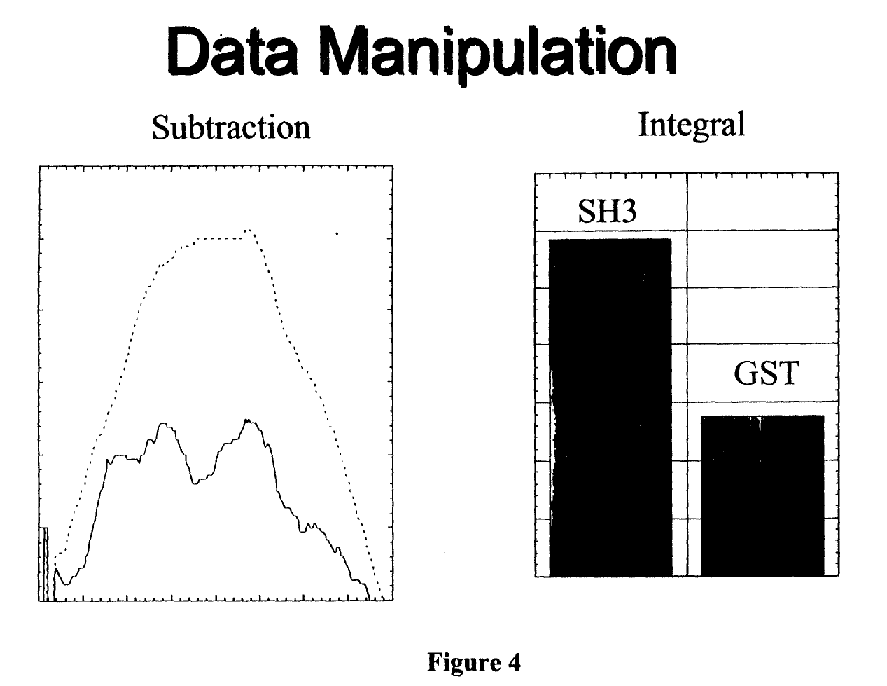

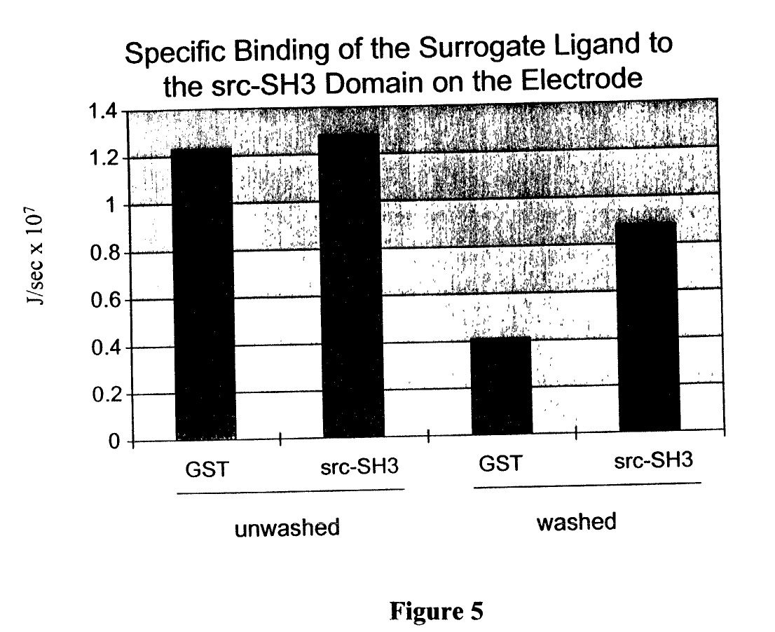

The electrochemical analysis apparatus and methods of the invention were used to detect and analyze the specific binding interaction between the Src SH3 domain and a specific binding SH3 peptide as follows. Electrodes prepared as described in Example 2 were coated with a glutathione sulfur transferase (GST)-Src SH3 fusion protein (prepared using the GST Gene Fusion system, obtained from Pharmacia), or GST itself. The NPDF-1 peptide (GHGSGRALPPLPRY; SEQ ID No.: 1) was labeled with ruthenium, as described above in Example 1 and shown in FIG. 6. The electrodes were incubated with a solution of the labeled peptide and a ruthenium mediator (hexaamineruthenium(III)) for 2 hours. The electrodes were washed with buffer and cyclic voltammetry performed as described in Example 3. The assays were also performed in the presence of the ruthenium electrochemical mediator and in the absence of ruthenium-labeled peptide. Data analysis was performed by integration of the cyclic voltammetric curves (as shown in FIG. 3) and subtraction of the background signal produced by incubation and cyclic voltammetry in the presence of the electrochemical mediator alone (as shown in FIG. 4). The area under the voltammogram curves was integrated using an integration program, and the integrated current data obtained in the presence of mediator alone was subtracted from the data obtained in the presence of the electrochemically-labeled probe for electrodes coated with GST alone and with the GST-Src-SH3 fusion protein. These results revealed a much higher signal produced by the interaction of the peptide with the GST-Src SH3 coated electrode (signal analysis shown in FIG. 5). These results demonstrated that the electrochemical analytical assay of the invention was capable of detecting specific protein-peptide interactions.

The interaction of the electrochemically-labeled SH3-binding peptide is shown to be time-dependent using the electrochemical analysis assays of the invention. To demonstrate this time dependency, electrodes coated with the GST-Src SH3 fusion protein or GST are incubated with excess electrochemically-labeled SH3 binding peptide, prepared as described in Example 1. Electrochemical measurements using cyclic voltammetry are obtained at 30 minute intervals over a period of at least 4 hours. An increase in the voltammetric signal (comprising the difference between the integrated current in the presence and absence of the electrochemically-labeled peptide) is detected over the time course of the experiment using the GST-Src SH3 fusion protein-immobilized electrode, until a plateau is reached at saturating concentrations of the electrochemically-labeled specific binding peptide.

The dependency of the electrochemical signal on the concentration of the electrochemically-labeled peptides of the invention is demonstrated using the GST-Src SH3-SH3 binding peptide binding pair. In these experiments, varying amounts of the GST-Src SH3 fusion protein are immobilized to electrodes as described in Example 2. For each GST-Src SH3 immobilized electrode, cyclic voltammetry is performed using a range of electrochemically-labeled SH3-binding peptide concentrations analyzed for the production of an electrochemical signal after a particular incubation time. The signal for each concentration of immobilized GST-Src SH3 increases with increasing peptide concentration, and for equivalent peptide concentrations the voltammetric signal increases with increasing amounts of target fusion protein immobilized on the electrode. These results demonstrate that the magnitude of the electrochemical signal is proportional to the concentration of the immobilized protein on the electrode, as well as the concentration of the electrochemically-labeled peptide in the solution.

Specific protein-peptide interaction is also demonstrated by electrochemical analysis of the interaction between streptavidin and a peptide that binds to the biotin binding site. Electrodes are prepared by coating with the GST-Src SH3 fusion protein described above or with streptavidin using the procedures described in Example 2. The coated electrodes are then incubated in the presence of the appropriate electrochemical mediator and an electrochemically-labeled species of Src SH3 binding peptide as above or with an electrochemically-labeled species of a peptide having the amino acid sequence:

His-Gly-Ser-Gly-Ser-Phe-Ser-His-Pro-Gln-Asn-Thr (SEQ ID No.:2)

that binds to the biotin binding site on streptavidin. After this incubation the electrodes are washed and cyclic voltammetry performed as described in Example 3. Cyclic voltammetry data is integrated and the background integrated current obtained using these electrodes in the presence of electrochemical mediator and in the absence of the electrochemically-labeled specific peptides subtracted therefrom as described above. Specific signals are detected for electrodes having immobilized GST-Src SH3 and analyzed in the presence of the electrochemically-labeled SH3 binding peptide, as well as for electrodes having immobilized streptavidin in the presence of the electrochemically-labeled streptavidin binding peptide. However, no specific signal is detected for electrodes having immobilized GST-Src SH3 and analyzed in the presence of the electrochemically-labeled streptavidin binding peptide, or for electrodes having immobilized streptavidin in the presence of electrochemically-labeled SH3 binding peptide.

Another demonstration of the use of the electrochemical analysis assays of the invention is performed to detect specific interaction between an electrode having the immobilized target protein MDM2 and an electrochemically-labeled peptide corresponding to a fragment of the tumor suppressor gene, p53. Electrodes coated as described above in Example 2 with the target protein MDM2 is used capture a surrogate ligand having a amino acid sequence derived from the native amino acid sequence of p53:

biotin-His-His-Ser-Gly-Ser-Gly-Ser-Gln-Thr-Phe-Ser-Asp-Leu-Trp-Lys-Leu (SEQ ID No.: 3),

which is known to specifically bind to MDM2 (see, Picksley et al., 1994, Oncogene 9: 2523). Electrodes coated with the GST-Src SH3 fusion protein or streptavidin are used as controls for non-specific signal. MDM2-immobilized electrodes are incubated in the presence of electrochemically-labeled p53 peptide. The electrodes are then washed and cyclic voltammetry performed in the presence of an electrochemical mediator as described above. Cyclic voltammetry data is integrated and the background integrated current obtained using these electrodes in the presence of mediator and in the absence of the electrochemically-labeled specific peptides subtracted therefrom as described above. Specific signals are detected in cyclic voltammetry experiments using MDM2-immobilized electrodes in the presence of electrochemically-labeled p53 peptide and mediator, indicative of a specific interaction between the p53 peptide and MDM2. No specific interaction is detected in cyclic voltammetry experiments using the electrochemically-labeled p53 peptide and electrodes having immobilized GST-Src SH3 or streptavidin.

These results show that the immobilized electrodes of the invention can be used to detect specific interactions between biological binding pairs comprising proteins and electrochemically-labeled specific binding peptides.

### EXAMPLE 5

### Electrochemical Assay for Screening Combinatorial Libraries

The electrochemical assay of the invention was used to screen combinatorial chemical libraries to detect samples that perturb the electrochemical signal obtained from the interaction between the GST-Src-SH3 fusion protein and electrochemically-labeled SH3 binding peptide by cyclic voltammetry. For the electrochemical assay to be useful for screening libraries of chemical compounds for compounds that disrupt the target:peptide interaction, the conditions of the screen must not be easily perturbed, or the cyclic voltammetry output diminished thereby. Optimally, such a screen should function over a wide range of pH and salt concentrations, and be insensitive to common contaminants (such as coupling reagents) that are frequently encountered in combinatorial chemical libraries.

To evaluate the robustness of the electrochemical analysis assay for screening combinatorial libraries under various conditions, GST-Src SH3-immobilized electrodes of the invention are incubated with electrochemically-labeled SH3 binding peptide, as described above. Cyclic voltammetry experiments are performed in the presence of selected chemicals such as acids, bases, salts, and chemicals containing functional groups such as aldehydes, ketones, and alcohols. In these experiments, the presence of most of these chemicals is found to have no effect on the electrochemical signal.

### EXAMPLE 6

### Electrochemical Analysis for Determining the K_{d} for a Known Protein:Peptide Interaction

The electrochemical analysis methods of the invention are used to determine the K_{d} of the interaction between the Src-SH3 domain and a number of short, proline-rich specific binding peptides. The interaction of the Src SH3 domain with short, proline-rich peptides such as Arg-Pro-Leu-Pro-Pro-Leu-Pro (SEQ ID No.: 4) and Ala-Pro-Pro-Val-Pro-Pro-Arg (SEQ ID No.: 5) have been intensively studied, and K_{d} values have been determined by validated means such as BIAcore® (Pharmacia). On average, these peptides have been shown to bind to the Src SH3 domain with a K_{d} of 5 μM. These data provide a solid basis for comparison of the ability of the electrochemical assay to provide an accurate K_{d} value for the same interaction.

The K_{d} value for the interaction of GST-Src SH3 and SH3 binding peptides is determined using the electrochemical analysis methods of the invention to provide a comparison with a pharmacologically-validated method. Electrodes coated with the GST-Src SH3 fusion protein are incubated with electrochemically-labeled species of the proline-rich SH3-domain specific binding peptides shown above. Electrochemical signal is generated using cyclic voltammetry as described above, and the signal is monitored over time as described in Example 5 above. Electrochemical signal data is collected at various concentrations of the peptide, and the electrochemical signal used to calculate a K_{d} value for the peptide. K_{d} values are also determined using the BIAcore® method as an internal control, and a comparison of the results between the two analytical methods are used to validate the values determined using the electrochemical analysis assay of the invention.

### EXAMPLE 7

### Electrochemical Analysis for Detecting Protein:Peptide Interactions in Complex Mixtures

The electrochemical analytic methods and apparati of the invention are used to detect protein:peptide interactions in a complex mixture. A variety of different target molecules and purchased from Polysciences (Warrington, Pa.). The redox polymer poly(1-vinylimidazole), modified with Os bipyridine redox centers (PVI-Os) was synthesized as described by Ohara et al. (1993, Anal. Chem. 65: 3512-3517). Specifically, hydrogels were prepared by mixing together 5 μL of each of the following solutions: a solution of a first member of a biological binding pair, typically comprising a receptor protein or fragment at a concentration of 4-6 mg/mL protein; 10 mg/mL PVI-Os, and 2.5 mg/mL PEGDGE. A 1 μL aliquot of the mixture was then spread on the surface of the glassy carbon electrodes. The hydrogel coated electrodes were cured overnight at room temperature prior to use.

### B. Electrochemical Detection of Surrogate Ligand Binding to src SH3 Using a Hydrogel

The src SH3 domain was immobilized in a hydrogel as described above in Section A. This electrode was then used for electrochemical detection of surrogate ligand binding using a surrogate ligand prepared as follows.

A complex of streptavidin (SA) (Sigma Chemical Co., St. Louis, Mo.), biotinylated horseradish peroxidase (B-HRP) (Sigma) and biotinylated src SH3 surrogate ligand (His-Gly-Ser-Gly-Ser-Phe-Ser-His-Pro-Gln-Asn-Thr; SEQ ID No. 2) was prepared as follows. Biotinylated src SH3 surrogate ligand (3 μL of a 120 μM (4 mg/mL) solution, 400 pmol) was mixed with B-HRP (4 μL of a 25 μM solution (1 mg/mL), 100 pmol) and the mixture was transferred to a tube containing 16 μg SA (17 μL of a 16 μM (1 mg/mL) solution). This mixture was incubated undisturbed at room temperature for 20 minutes. Biotin (25 μL of a 100 μM solution, 250 pmol) was then added and the solution volume was increased to 100 μL with phosphate buffered saline (PBS) solution.

Electrochemical analysis (chronoamperometry) was conducted using a BAS100B electrochemical analyzer (BAS, West Lafayette, Ind.). The src SH3-hydrogel coated electrode described above, a Ag/AgCl reference electrode (BAS) and a platinum auxiliary electrode were immersed in a 5 mL solution of PBS containing 1% bovine serum albumin. The solution was stirred throughout the course of the electrochemical analysis. The potential between the src SH3-hydrogel coated (working) electrode and the reference electrode was set at 100 mV. At t=300 sec, the solution was brought to a final concentration of 100 μM H₂O₂ by the addition of 5 μL of a 0.1M hydrogen peroxide solution. At t=600 sec, 31 μL of the streptavidin/biotinylated horse radish peroxidase (SA/B-HRP) conjugated surrogate ligand was added to a final concentration of 1 μg/mL SA.

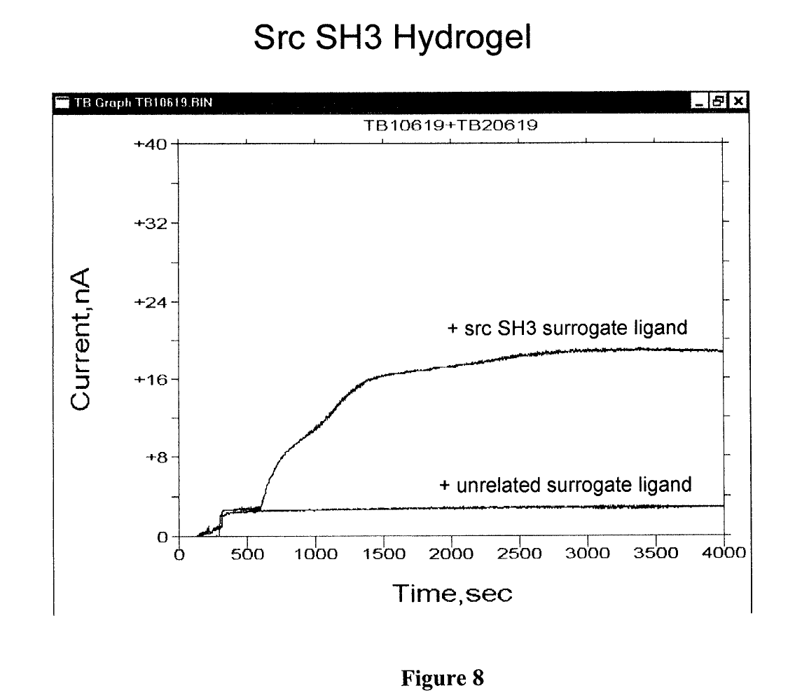

The results of this experiment are shown in FIG. 8, which is a graph of the current (nA) produced by the sequential addition of these components to the electrochemical analysis solution. As shown in FIG. 8, an increase in current was detected over time upon addition of the src SH3 surrogate ligand. This current was produced immediately upon addition of the SA/B-HRP conjugated surrogate ligand, reaching a plateau at about 1500-2000 sec. As a negative control, an unrelated surrogate ligand, prepared as described above, was used in the chronoamperometry experiment. No current was detected using this surrogate ligand.

These results demonstrated that the use of hydrogel electrodes containing a first member of a biological binding pair and an electrochemical mediator immobilized thereon could be used with a conjugate of a second member of a biological binding pair and a redox-dependent enzymatic catalyst in the presence of its substrate to detect binding between the members of biological binding pair using chronoamperometry.

### C. Electrochemical Detection of Surrogate Ligand Binding to tyrRS

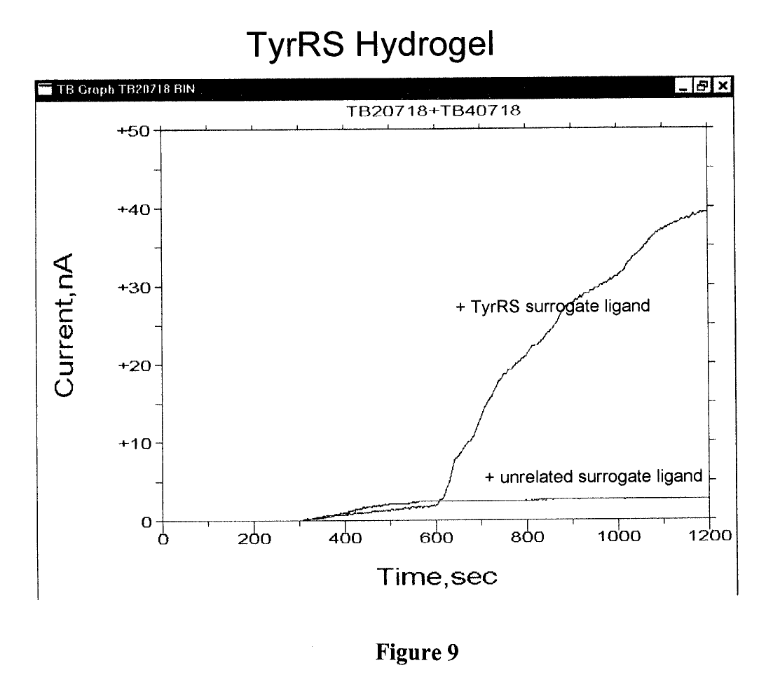

Tyrosine aminoacyl tRNA synthetase (tyrRS) was immobilized in a hydrogel as described in Section A. A complex containing the tyrRS surrogate ligand was prepared as described in Section B for the src SH3 surrogate ligand. Chronoamperometry was conducted as described in Section B and the results of these experiments are shown in FIG. 9. The tyrRS surrogate ligand had the amino acid sequence:

Leu-Tyr-Ser-Trp-Pro-Asp-Glu-Gln-Tyr-Glu-Arg-Pro-Ser-Gly-Ser-Gly-Lys (SEQ ID No.: 6).

As seen with the src SH3-SA/B-HRP conjugated surrogate ligand experiments, current was detected in experiments using the tyrRS-SA/B-HRP conjugated surrogate ligand binding pair only when the conjugated surrogate ligand was added to the electrolyte solution. Negative control experiments using SA/B-HRP conjugates of an unrelated surrogate ligand produced no detectable increase in current under these experimental conditions.

These results confirmed the results described in Section B, and illustrated the generality of this experimental approach for electrochemical detection of binding between biological binding pairs.

### EXAMPLE 9

### Electrochemical Detection of Inhibitors of Biological Pair Binding

Electrochemical analysis of compounds for the capacity to inhibit specific binding between members of biological binding pairs was performed using the electrochemical analysis apparati and methods described in Example 8.

There were three alternate embodiments of the methods of the invention for detecting an inhibitor of biological pair binding as described in Example 8. In the first embodiment, inhibitor was added to the electrolyte solution after the conjugated surrogate ligand had bound to the target in the hydrogel; this was accomplished by adding the conjugated surrogate ligand to the electrolyte solution, detecting current generation until the plateau current was reached, adding a putative inhibitor and detecting a decrease in the amount of current produced. In the second embodiment, the inhibitor and the conjugated surrogate ligand were added to the electrolyte solution concurrently, and the amount of current produced in the presence of the putative inhibitor compared with the amount of current produced in its absence. In the third embodiment, inhibitor was added to the electrolyte solution prior to addition of the conjugated surrogate ligand.

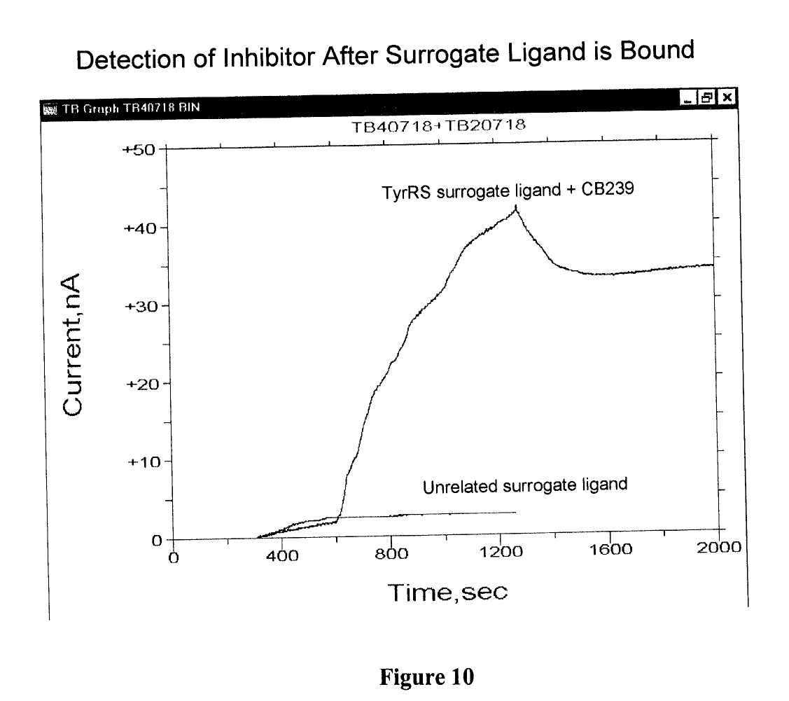

The results of analyses using the first method are shown in FIG. 10. Hydrogel electrodes containing tyrRS and the SA/B-HRP conjugated tyrRS surrogate ligand were prepared as described in Example 8. Chronoamperometry was conducted as described in Example 8 with the exception that at t=1300 sec, the electrolyte solution was brought to a final concentration of 10 μM of a specific inhibitor of tyrRS, NL932, by the addition of 100 μL of a 1 mM solution of the inhibitor. As shown in FIG. 10, addition of the inhibitor produced a sudden drop in the current (about a 25% drop in produced current). These results indicate that inhibitors of biological binding pair binding can be detected was achieved using the hydrogel electrodes of the invention even after binding of the biological binding pair.

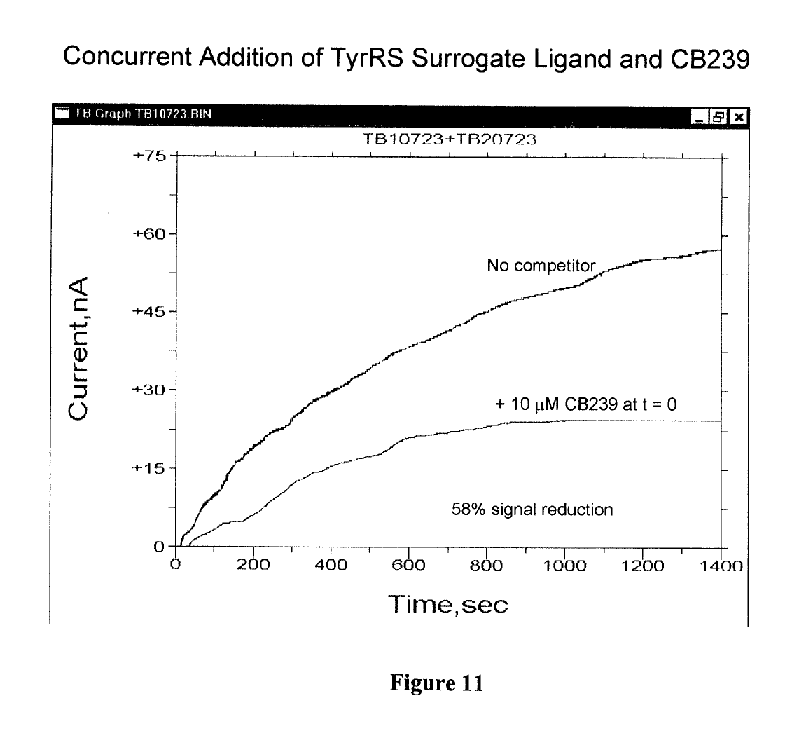

The results of analyses using the second method are shown in FIG. 11. Hydrogels containing tyrRS and SA/B-HRP conjugates of the tyrRS surrogate ligand were prepared as described in Example 8. Chronoamperometry was conducted as described in Example 8 with the exception that 10 μM NL932 (final concentration, added as described above) was added to the electrolyte solution at the same time as the surrogate ligand (at about t=0). As shown in FIG. 11, concurrent addition of inhibitor and conjugated surrogate ligand resulted in a 58% reduction in the peak current. The observed reduction using this method was greater that the reduction observed using the first method described above.

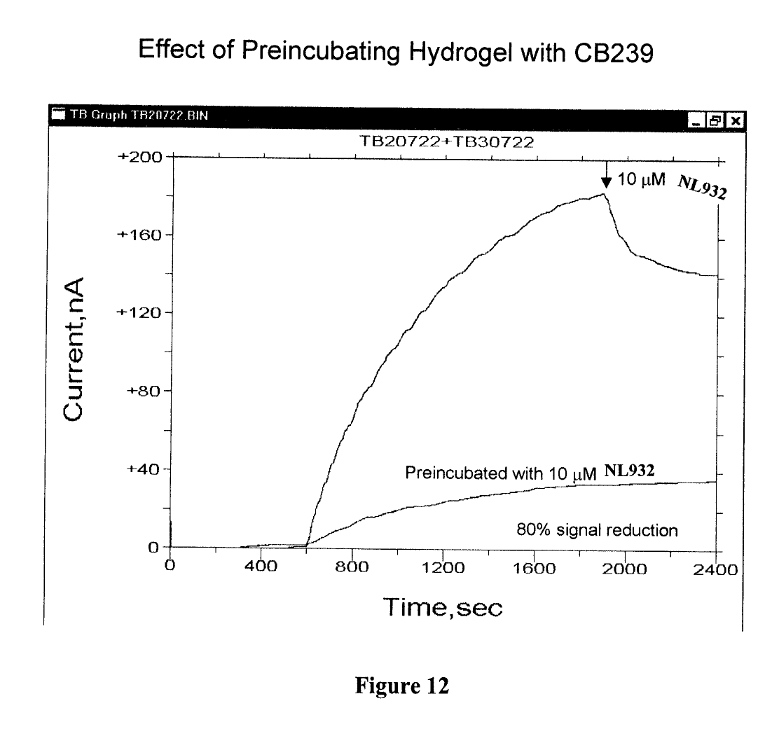

The results of analyses using the third method are shown in FIG. 12. Hydrogels containing tyrRS and SA/B-HRP conjugates of the tyrRS surrogate ligand were prepared as described in Example 8. Chronoamperometry was conducted as described in Example 8 with the exception that the hydrogel electrode was incubated with stirring in 5 mL solution containing 10 μM NL932 for 15 minutes prior to initiation of chronoamperometry. The chronoamperometry experiment was initiated by the addition of the conjugated tyrRS surrogate ligand and substrate; the electrode was maintained in the inhibitor solution for an additional 10 minutes prior to the addition of the conjugated surrogate ligand. As shown in FIG. 12, preincubation of the hydrogel with the inhibitor produced an 80% reduction in the peak current when compared with chronoamperometry performed in the absence of inhibitor.

While all three methods described were found to permit the detection of a tyrRS inhibitor, the most sensitive method was determined to be preincubation of the inhibitor with hydrogel (the third described method). Concurrent addition of the inhibitor and surrogate ligand was intermediate in sensitivity (the second described method), and addition of the inhibitor following the surrogate ligand was the least sensitive (the first described method). These results demonstrated the capacity of the methods and apparati of the invention to provide for the sensitive detection of compounds that inhibit the binding of biological binding pairs, thereby providing a sensitive screening method for drug leads directed towards disrupting inappropriate or pathological biological binding pair binding associated with disease.

### EXAMPLE 10

### Electrochemical Measurement of Binding Rate Constants using Surrogate Ligands

Chronoamperometric analysis of binding assays between tyrRS and its surrogate ligand as disclosed in Example 8, Section C was used to determine binding rate constants of the surrogate ligand to tyrRS immobilized on an electrode according to the invention. Hydrogels containing tyrRS were prepared as described in Example 8, Section A. The complex containing the tyrRS surrogate ligand was prepared, and chronoamperometry was performed as described in Example 8, Section C. As shown in FIG. 9, there was a dramatic increase observed in current upon addition of the surrogate ligand complex. The current obtains a saturation-limited value according to single-exponential rate kinetics. The first-order rate constants for binding or displacement of the surrogate ligand complex can be calculated according to the equation:

(Iₛₐₜ−I)=(Iₛₐₜ−I₀)exp^{(-kt)}

where I is current at time t, k is the first order rate constant, and the subscripts “o” and “sat” denote values observed at the time of complex addition and upon saturation of binding, respectively. The first order rate constant can be determined using the relation:

k=−{ln(I−Iₒ)−ln(Iₛₐₜ−I₀)}/t

This equation was used to determine a binding rate constant for the tyrRS-surrogate ligand binding reaction of 0.0012 s⁻¹.

Similarly, addition of an inhibitor of binding to the tyrRS-surrogate ligand results in a reduction of current, as shown in FIG. 10 and described in Example 9. This reduction of current also achieves a saturation-limited value that can be used to determine the rate constant for displacement of the surrogate ligand by the inhibitor, using the above equations. The binding rate constant for displacement of the surrogate ligand was determined to be 0.0035 s⁻¹ for the competitive inhibitor NL932.

### EXAMPLE 11

### Determination of Inhibition Constant of a Competitive Inhibitor Against a Surrogate Ligand Complex

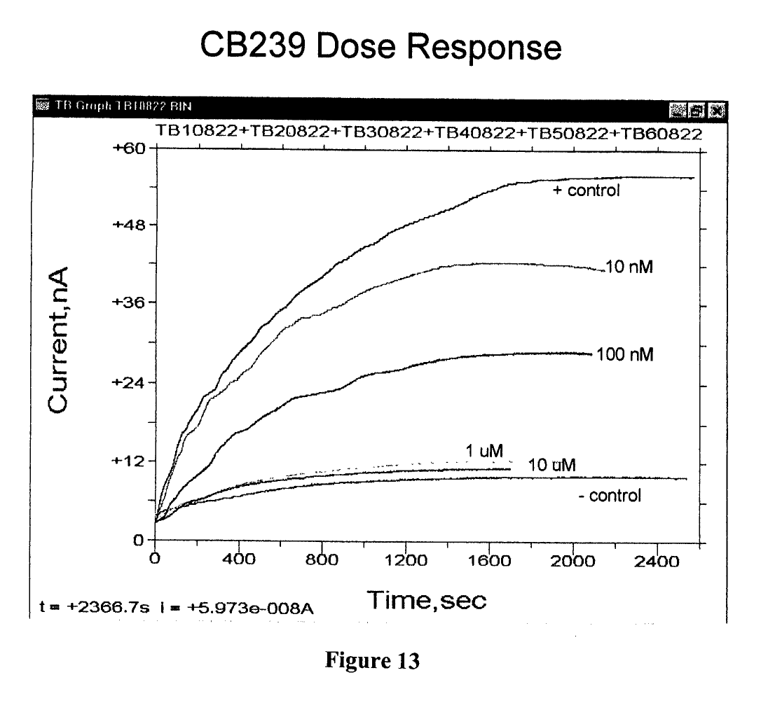

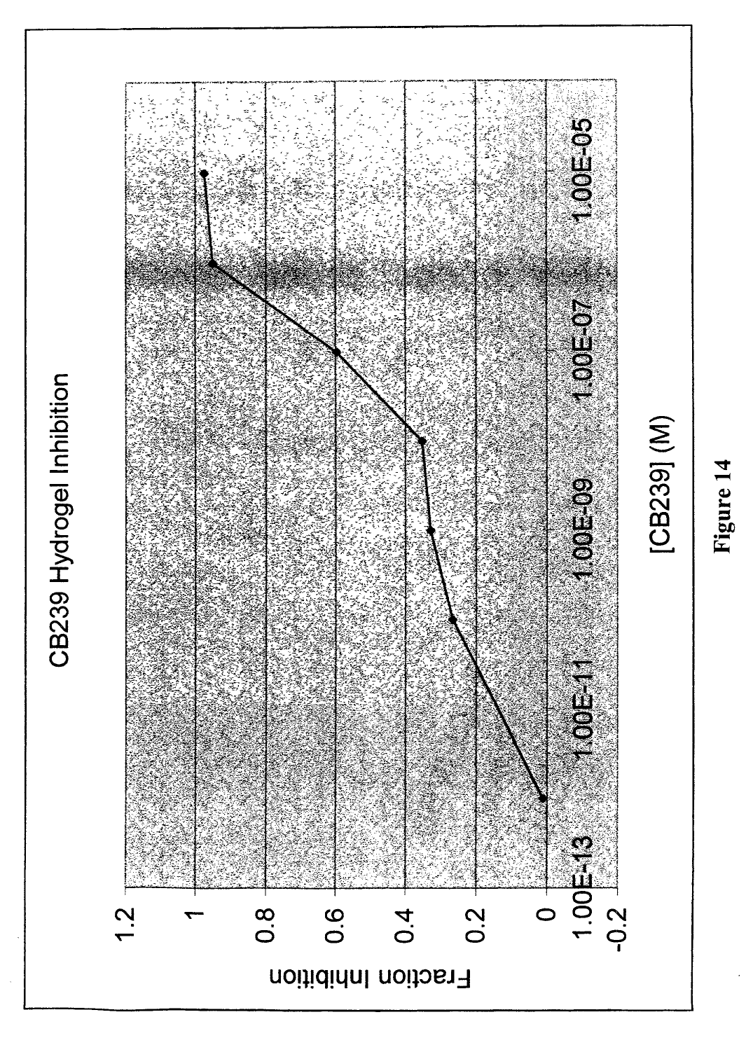

Chronoamperometric analysis of binding assays between tyrRS and its surrogate ligand as disclosed in Example 8, Section C was used to determine the inhibition constant of a competitive inhibitor of surrogate ligand binding to tyrRS immobilized on an electrode according to the invention. Hydrogels containing tyrRS were prepared as described in Example 8, Section A. The complex containing the tyrRS surrogate ligand was prepared, and chronoamperometry was performed as described in Example 8, Section C. As shown in FIG. 13, addition of increasing concentrations of competitive inhibitor NL932 to the electrode-immobilized tyrRS-surrogate ligand complex suppressed specific binding of the surrogate ligand to tyrRS. A graph of the fraction inhibited versus inhibitor concentration is shown in FIG. 14, and indicates that competitive inhibition onset occurred at an inhibitor concentration of approximately 50 mM. Similar analysis of other competitive inhibitors can be used to determine the specific inhibition constant for each competitive inhibitor of tyrRS-surrogate ligand complex binding.

It should be understood that the foregoing disclosure emphasizes certain specific embodiments of the invention and that all modifications or alternatives equivalent thereto are within the spirit and scope of the invention as set forth in the appended claims.

What is claimed is:

1. An apparatus for performing an electrochemical assay for detecting binding between members of a biological binding pair, the apparatus comprising
a first electrode, wherein the electrode comprises a conducting or semiconducting material, and wherein the electrode has a surface that is coated with a porous, hydrophilic, polymeric layer to which a first member of the biological binding pair is immobilized thereto,
a second, reference electrode comprising a conducting metal in contact with an aqueous electrolyte solution,
a third, auxiliary electrode comprising a conducting metal
wherein each of the electrodes is electrically connected to a potentiostat, and wherein the apparatus further comprises
a reaction chamber containing a solution of an electrolyte, wherein each of the electrodes is in electrochemical contact therewith, the solution further containing
an electrochemical mediator comprising a chemical species capable of participating in a reduction/oxidation reaction with the electrodes under conditions whereby an electrical potential is applied to the electrodes, and wherein the solution further contains
a second member of the biological binding pair, wherein said second member is electrochemically labeled with a chemical species capable of participating in a reduction/oxidation reaction with the electrochemical mediator under conditions whereby an electrical potential is applied to the electrodes
wherein a current is produced in the apparatus when an electrical potential is applied to the electrodes under conditions wherein the second member of the biological binding pair is bound to the first member of the biological binding pair.

2. An apparatus according to claim 1, wherein the electrochemical assay is cyclic voltammetry.

3. An apparatus according to claim 1, wherein the apparatus further comprises a multiplicity of each of the electrodes and a multiplicity of reaction chambers, wherein each reaction chamber contains an electrolyte and is in electrochemical contact with one each of the three electrodes among the multiplicity of electrodes in the apparatus.

4. An apparatus according to claim 1, wherein the second member of the biological binding pair is electrochemically labeled with ruthenium.

5. An apparatus according to claim 1, wherein the electrochemical mediator is a ruthenium compound.

6. An apparatus according to claim 1, wherein the first member of the biological binding pair is a receptor protein or ligand binding fragment thereof and the second member of the biological binding pair is a ligand that specifically binds to the receptor protein.

7. An apparatus according to claim 1, wherein the first member of the biological binding pair is an antibody protein or antigen binding fragment thereof and the second member of the biological binding pair is an antigen that specifically binds to the antibody.

8. An apparatus according to claim 1, wherein the first member of the biological binding pair is a first protein or fragment thereof and the second member of the biological binding pair is a second protein or fragment thereof that specifically binds to the first protein.

9. An electrode comprising a conducting or semiconducting material, wherein the electrode has a surface that is coated with a porous, hydrophilic, polymeric layer to which a first member of a biological binding pair is immobilized thereto.

10. A kit for preparing an electrode according to claim 9, wherein the kit comprises an electrode comprising a conducting or semi-conducting material, a first member of a biological binding pair, a reagent for preparing a porous, hydrophilic, polymeric layer on the surface of the electrode, and a reagent for immobilizing the first member of the biological binding pair within the porous, hydrophilic, polymeric layer on the surface of the electrode.

11. A method for preparing an electrode according to claim 9, comprising the steps of:
a) providing an electrode comprising a conducting or semi-conducting material;
b) preparing a porous, hydrophilic, polymeric layer on the surface of the electrode; and
c) immobilizing a first member of a biological binding pair within the porous, hydrophilic, polymeric layer on the surface of the electrode.

12. An electrochemically labeled surrogate ligand comprising a second member of a biological binding pair having a binding affinity of from about 1 nanomolar (nM) to about 100 micromolar (μM) for a first member of a biological binding pair.

13. An electrochemically labeled surrogate ligand according to claim 12, labeled with a ruthenium compound.

14. An electrochemically labeled surrogate ligand according to claim 12, comprising a peptide of formula:
Gly-His-Gly-Ser-Gly-Arg-Ala-Leu-Pro-Pro-Leu-Pro-Arg-Tyr-NH₂ (SEQ ID No.: 1);
His-Gly-Ser-Gly-Ser-Phe-Ser-His-Pro-Gln-Asn-Thr (SEQ ID No.: 2);
biotin-His-His-Ser-Gly-Ser-Gly-Ser-Gln-Thr-Phe-Ser-Asp-Leu-Trp-Lys-Leu (SEQ ID No.: 3),
Arg-Pro-Leu-Pro-Pro-Leu-Pro (SEQ ID No.: 4),
Ala-Pro-Pro-Val-Pro-Pro-Arg (SEQ ID No.: 5),
or
Leu-Tyr-Ser-Trp-Pro-Asp-Glu-Gln-Tyr-Glu-Arg-Pro-Ser-Gly-Ser-Gly-Lys (SEQ ID No.: 6)

15. An electrochemical mediator according to claim 1 comprising a ruthenium compound.

16. A method for detecting binding of an electrochemically labeled second member of a biological binding pair with a first member of a biological binding pair immobilized on an electrode using an apparatus according to claim 1, the method comprising the steps of:
a) providing a first reaction chamber in electrochemical contact with each of the electrodes according to claim 1, wherein the first electrode comprises a first member of the biological binding pair immobilized thereto, and a second reaction chamber in electrochemical contact with each of the electrodes according to claim 1, wherein the first electrode comprises a first member of the biological binding pair immobilized thereto, each of the electrodes being electrically connected to a potentiostat;
wherein the first reaction chamber contains an electrochemical mediator according to claim 1 and an electrochemically-labeled second member of the biological binding pair that specifically binds to the immobilized first member of the biological binding pair, and wherein the second reaction chamber contains an electrochemical mediator according to claim 1 and an electrochemically-labeled species that does not specifically bind to the immobilized first member of the biological binding pair; the method further comprising the steps of:
b) performing an electrochemical assay in each of the first and second reaction chambers of the apparatus of claim 1 to produce a current in the electrodes of the apparatus; and
c) comparing the current produced in the electrochemical assay in the first reaction chamber to the current produced in the electrochemical assay in the second reaction chamber
wherein binding of the electrochemically labeled second member of the biological binding pair with the immobilized first member of the biological binding pair is detected by the production of a larger current in the first reaction chamber than is produced in the second reaction chamber.

17. A method for identifying an inhibitor of binding of an electrochemically labeled second member of a biological binding pair with a first member of a biological binding pair immobilized on an electrode using an apparatus according to claim 1, the method comprising the steps of:
a) providing a first reaction chamber in electrochemical contact with each of the electrodes according to claim 1, wherein the first electrode comprises a first member of the biological binding pair immobilized thereto, and a second reaction chamber in electrochemical contact with each of the electrodes according to claim 1, wherein the first electrode comprises a first member of the biological binding pair immobilized thereto, each of the electrodes being electrically connected to a potentiostat;
wherein each of the reaction chambers contains an electrochemical mediator according to claim 1 and an electrochemically-labeled second member of the biological binding pair that specifically binds to the immobilized first member of the biological binding pair, and wherein the second reaction chamber further contains an inhibitor of binding of the second member of the biological binding pair that specifically binds to the immobilized first member of the biological binding pair; the method further comprising the steps of:
b) performing an electrochemical assay in each of the first and second reaction chambers of the apparatus of claim 1 to produce a current in the electrodes of the apparatus; and
c) comparing the current produced in the electrochemical assay in the first reaction chamber to the current produced in the electrochemical assay in the second reaction chamber
wherein an inhibitor of binding of the electrochemically labeled second member of the biological binding pair with the immobilized first member of the biological binding pair is identified by the production of a larger current in the first reaction chamber than is produced in the second reaction chamber.

18. A method for screening a complex chemical mixture for an inhibitor of binding of an electrochemically labeled second member of a biological binding pair with a first member of a biological binding pair immobilized on an electrode using an apparatus according to claim 1, the method comprising the steps of:
a) providing a first reaction chamber in electrochemical contact with each of the electrodes according to claim 1, wherein the first electrode comprises a first member of the biological binding pair immobilized thereto, and a second reaction chamber in electrochemical contact with each of the electrodes according to claim 1, wherein the first electrode comprises a first member of the biological binding pair immobilized thereto, each of the electrodes being electrically connected to a potentiostat;
wherein each of the reaction chambers contains an electrochemical mediator according to claim 1 and an electrochemically-labeled second member of the biological binding pair that specifically binds to the immobilized first member of the biological binding pair, and wherein the second reaction chamber further contains a portion of the complex mixture comprising an inhibitor of binding of the second member of the biological binding pair that specifically binds to the immobilized first member of the biological binding pair; the method further comprising the steps of:
b) performing an electrochemical assay in each of the first and second reaction chambers of the apparatus of claim 1 to produce a current in the electrodes of the apparatus; and
c) comparing the current produced in the electrochemical assay in the first reaction chamber to the current produced in the electrochemical assay in the second reaction chamber
wherein a complex mixture having an inhibitor of binding of the electrochemically labeled second member of the biological binding pair with the immobilized first member of the biological binding pair is identified by the production of a larger current in the first reaction chamber than is produced in the second reaction chamber.

19. A method according to claim 16 wherein the second member of the biological binding pair is an electrochemically labeled surrogate ligand.

20. A method according to claim 17 wherein the second member of the biological binding pair is an electrochemically labeled surrogate ligand.

21. A method according to claim 18 wherein the second member of the biological binding pair is an electrochemically labeled surrogate ligand.

22. A method according to 18 comprising the additional steps of:
d) chemically fractionating the complex mixture having an inhibitor of binding of the second member of the biological binding pair to the first member of the biological binding pair immobilized on the first electrode, to produce fractionated submixtures; and
e) performing steps (a) through (c) of the method of claim 18 on each of the fractionated submixtures to identify the submixtures that have an inhibitor of binding of the biological binding pair.

23. An apparatus for performing an electrochemical assay for detecting binding between members of a biological binding pair, the apparatus comprising
a first electrode, wherein the electrode comprises a conducting or semiconducting material, and wherein the electrode has a surface that is coated with a porous, hydrophilic, polymeric layer, wherein a first member of the biological binding pair and an electrochemical mediator comprising a chemical species capable of participating in a reduction/oxidation reaction with the electrodes under conditions whereby an electrical potential is applied to the electrodes, are each immobilized thereto,
a second, reference electrode comprising a conducting metal in contact with an aqueous electrolyte solution,
a third, auxiliary electrode comprising a conducting metal
wherein each of the electrodes is electrically connected to a potentiostat, and wherein the apparatus further contains
a reaction chamber containing a solution of an electrolyte, wherein each of the electrodes is in electrochemical contact therewith, the solution further comprising
a second member of the biological binding pair, wherein said second member is electrochemically labeled with a chemical species capable of participating in a reduction/oxidation reaction with the electrochemical mediator under conditions whereby an electrical potential is applied to the electrodes
wherein a current is produced in the apparatus when an electrical potential is applied to the electrodes under conditions wherein the second member of the biological binding pair is bound to the first member of the biological binding pair.

24. An apparatus according to claim 23, wherein the electrochemical assay is cyclic voltammetry.

25. An apparatus according to claim 23, wherein the apparatus further comprises a multiplicity of each of the electrodes and a multiplicity of reaction chambers, wherein each reaction chamber contains an electrolyte and is in electrochemical contact with one each of the three electrodes among the multiplicity of electrodes in the apparatus.

26. An apparatus according to claim 23, wherein the second member of the biological binding pair is electrochemically labeled with ruthenium.

27. An apparatus according to claim 23, wherein the electrochemical mediator is an osmium compound.

28. An apparatus according to claim 23, wherein the electrochemical mediator is a ruthenium compound.

29. An apparatus according to claim 23, wherein the first member of the biological binding pair is a receptor protein or ligand binding fragment thereof and the second member of the biological binding pair is a ligand that specifically binds to the receptor protein.

30. An apparatus according to claim 23, wherein the first member of the biological binding pair is an antibody protein or antigen binding fragment thereof and the second member of the biological binding pair is an antigen that specifically binds to the antibody.

31. An apparatus according to claim 23, wherein the first member of the biological binding pair is a first protein or fragment thereof and the second member of the biological binding pair is a second protein or fragment thereof that specifically binds to the first protein.

32. An electrode comprising a conducting or semiconducting material, wherein the electrode has a surface that is coated with a porous, hydrophilic, polymeric layer and wherein a first member of the biological binding pair and an electrochemical mediator comprising a chemical species capable of participating in a reduction/oxidation reaction with the electrode under conditions whereby an electrical potential is applied to the electrode, are each immobilized thereto.

33. A kit for preparing an electrode according to claim 32, wherein the kit comprises an electrode comprising a conducting or semi-conducting material, an electrochemical mediator, a first member of a biological binding pair, a reagent for preparing a porous, hydrophilic, polymeric layer on the surface of the electrode, and a reagent for immobilizing the first member of the biological binding pair and the electrochemical mediator within the porous, hydrophilic, polymeric layer on the surface of the electrode.

34. A method for preparing an electrode according to claim 32, comprising the steps of:
a) providing an electrode comprising a conducting or semi-conducting material;
b) preparing a porous, hydrophilic, polymeric layer on the surface of the electrode; and
c) immobilizing a first member of a biological binding pair and the electrochemical mediator within the porous, hydrophilic, polymeric layer on the surface of the electrode.

35. A method for detecting binding of an electrochemically labeled second member of a biological binding pair with a first member of a biological binding pair immobilized on an electrode using an apparatus according to claim 23, the method comprising the steps of:
a) providing a first reaction chamber in electrochemical contact with each of the electrodes according to claim 23, wherein the first electrode comprises a first member of the biological binding pair immobilized thereto, and a second reaction chamber in electrochemical contact with each of the electrodes according to claim 23, wherein the first electrode comprises a first member of the biological binding pair immobilized thereto, each of the electrodes being electrically connected to a potentiostat;
wherein the first reaction chamber contains an electrochemically-labeled second member of the biological binding pair that specifically binds to the immobilized first member of the biological binding pair, and wherein the second reaction chamber contains an electrochemically-labeled species that does not specifically bind to the immobilized first member of the biological binding pair; the method further comprising the steps of:
b) performing an electrochemical assay in each of the first and second reaction chambers of the apparatus of claim 23 to produce a current in the electrodes of the apparatus; and
c) comparing the current produced in the electrochemical assay in the first reaction chamber to the current produced in the electrochemical assay in the second reaction chamber
wherein binding of the electrochemically labeled second member of the biological binding pair with the immobilized first member of the biological binding pair is detected by the production of a larger current in the first reaction chamber than is produced in the second reaction chamber.

36. A method for identifying an inhibitor of binding of an electrochemically labeled second member of a biological binding pair with a first member of a biological binding pair immobilized on an electrode using an apparatus according to claim 23, the method comprising the steps of:
a) providing a first reaction chamber in electrochemical contact with each of the electrodes according to claim 23, wherein the first electrode comprises a first member of the biological binding pair immobilized thereto, and a second reaction chamber in electrochemical contact with each of the electrodes according to claim 23, wherein the first electrode comprises a first member of the biological binding pair immobilized thereto, each of the electrodes being electrically connected to a potentiostat;
wherein each of the reaction chambers contains an electrochemically-labeled second member of the biological binding pair that specifically binds to the immobilized first member of the biological binding pair, and wherein the second reaction chamber further contains an inhibitor of binding of the second member of the biological binding pair that specifically binds to the immobilized first member of the biological binding pair; the method further comprising the steps of:
b) performing an electrochemical assay in each of the first and second reaction chambers of the apparatus of claim 23 to produce a current in the electrodes of the apparatus; and
c) comparing the current produced in the electrochemical assay in the first reaction chamber to the current produced in the electrochemical assay in the second reaction chamber
wherein an inhibitor of binding of the electrochemically labeled second member of the biological binding pair with the immobilized first member of the biological binding pair is identified by the production of a larger current in the first reaction chamber than is produced in the second reaction chamber.

37. A method for screening a complex chemical mixture for an inhibitor of binding of an electrochemically labeled second member of a biological binding pair with a first member of a biological binding pair immobilized on an electrode using an apparatus according to claim 23, the method comprising the steps of:
a) providing a first reaction chamber in electrochemical contact with each of the electrodes according to claim 23, wherein the first electrode comprises a first member of the biological binding pair immobilized thereto, and a second reaction chamber in electrochemical contact with each of the electrodes according to claim 23, wherein the first electrode comprises a first member of the biological binding pair immobilized thereto, each of the electrodes being electrically connected to a potentiostat;
wherein each of the reaction chambers contains an electrochemically-labeled second member of the biological binding pair that specifically binds to the immobilized first member of the biological binding pair, and wherein the second reaction chamber further contains a portion of the complex mixture comprising an inhibitor of binding of the second member of the biological binding pair that specifically binds to the immobilized first member of the biological binding pair; the method further comprising the steps of:
b) performing an electrochemical assay in each of the first and second reaction chambers of the apparatus of claim 23 to produce a current in the electrodes of the apparatus; and
c) comparing the current produced in the electrochemical assay in the first reaction chamber to the current produced in the electrochemical assay in the second reaction chamber
wherein a complex mixture having an inhibitor of binding of the electrochemically labeled second member of the biological binding pair with the immobilized first member of the biological binding pair is identified by the production of a larger current in the first reaction chamber than is produced in the second reaction chamber.

38. A method according to claim 35 wherein the second member of the biological binding pair is an electrochemically labeled surrogate ligand.

39. A method according to claim 36 wherein the second member of the biological binding pair is an electrochemically labeled surrogate ligand.

40. A method according to claim 37 wherein the second member of the biological binding pair is an electrochemically labeled surrogate ligand.

41. A method according to claim 37 comprising the additional steps of:
d) chemically fractionating the complex mixture having an inhibitor of binding of the second member of the biological binding pair to the first member of the biological binding pair immobilized on the first electrode, to produce fractionated submixtures; and
e) performing steps (a) through (c) of the method of claim 37 on each of the fractionated submixtures to identify the submixtures that have an inhibitor of binding of the biological binding pair.

42. An apparatus for performing an electrochemical assay for detecting binding between members of a biological binding pair, the apparatus comprising
a first electrode, wherein the electrode comprises a conducting or semiconducting material, and wherein the electrode has a surface that is coated with a porous, hydrophilic, polymeric layer, wherein a first member of the biological binding pair and an electrochemical mediator comprising a chemical species capable of participating in a reduction/oxidation reaction with the electrodes under conditions whereby an electrical potential is applied to the electrodes, are each immobilized thereto,
a second, reference electrode comprising a conducting metal in contact with an aqueous electrolyte solution,
a third, auxiliary electrode comprising a conducting metal
wherein each of the electrodes is electrically connected to a potentiostat, and wherein the apparatus further comprises
a reaction chamber containing a solution of an electrolyte, wherein each of the electrodes is in electrochemical contact therewith, the solution further comprising
a second member of the biological binding pair, wherein said second member is bound to an electrochemical catalyst capable of participating in a reduction/oxidation reaction with the electrochemical mediator under conditions whereby an electrical potential is applied to the electrodes, wherein the electrolyte in the reaction chamber further contains a substrate for the electrochemical catalyst
wherein a current is produced in the apparatus when an electrical potential is applied to the electrodes under conditions wherein the second member of the biological binding pair is bound to the first member of the biological binding pair in the presence of the substrate for the electrochemical catalyst bound to the second member of the biological binding pair.

43. An apparatus according to claim 42, wherein the electrochemical assay is chronoamperometry.

44. An apparatus according to claim 42, wherein the apparatus further comprises a multiplicity of each of the electrodes and a multiplicity of reaction chambers, wherein each reaction chamber contains an electrolyte and is in electrochemical contact with one each of the three electrodes among the multiplicity of electrodes in the apparatus.

45. An apparatus according to claim 42, wherein the electrochemical catalyst bound to the second member of the biological binding pair is an enzyme.

46. An apparatus according to claim 45, wherein the enzyme is horse radish peroxidase.

47. An apparatus according to claim 42, wherein the electrochemical mediator is an osmium compound.

48. An apparatus according to claim 42, wherein the first member of the biological binding pair is a receptor protein or ligand binding fragment thereof and the second member of the biological binding pair is a ligand that specifically binds to the receptor protein.

49. An apparatus according to claim 42, wherein the first member of the biological binding pair is an antibody protein or antigen binding fragment thereof and the second member of the biological binding pair is an antigen that specifically binds to the antibody.

50. An apparatus according to claim 42, wherein the first member of the biological binding pair is a first protein or fragment thereof and the second member of the biological binding pair is a second protein or fragment thereof that specifically binds to the first protein.

51. A method for detecting binding of an electrochemically labeled second member of a biological binding pair with a first member of a biological binding pair immobilized on an electrode using an apparatus according to claim 42, the method comprising the steps of:
a) providing a first reaction chamber in electrochemical contact with each of the electrodes according to claim 42, wherein the first electrode comprises a first member of the biological binding pair immobilized thereto, and a second reaction chamber in electrochemical contact with each of the electrodes according to claim 42, wherein the first electrode comprises a first member of the biological binding pair immobilized thereto, each of the electrodes being electrically connected to a potentiostat;
wherein the first reaction chamber contains a substrate for the electrochemical catalyst and a second member of the biological binding pair that specifically binds to the immobilized first member of the biological binding pair, bound to an electrochemical catalyst, and wherein the second reaction chamber contains a substrate for the electrochemical catalyst and a chemical species that does not specifically bind to the immobilized first member of the biological binding pair, bound to an electrochemical catalyst; the method further comprising the steps of:
b) performing an electrochemical assay in each of the first and second reaction chambers of the apparatus of claim 42 to produce a current in the electrodes of the apparatus; and
c) comparing the current produced in the electrochemical assay in the first reaction chamber to the current produced in the electrochemical assay in the second reaction chamber
wherein binding of the second member of the biological binding pair with the immobilized first member of the biological binding pair is detected by the production of a larger current in the first reaction chamber than is produced in the second reaction chamber.

52. A method for identifying an inhibitor of binding of an electrochemically labeled second member of a biological binding pair with a first member of a biological binding pair immobilized on an electrode using an apparatus according to claim 42, the method comprising the steps of:
a) providing a first reaction chamber in electrochemical contact with each of the electrodes according to claim 42, wherein the first electrode comprises a first member of the biological binding pair immobilized thereto, and a second reaction chamber in electrochemical contact with each of the electrodes according to claim 42, wherein the first electrode comprises a first member of the biological binding pair immobilized thereto, each of the electrodes being electrically connected to a potentiostat;
wherein each of the reaction chambers contains a substrate for the electrochemical catalyst and a second member of the biological binding pair that specifically binds to the immobilized first member of the biological binding pair, bound to an electrochemical catalyst, and wherein the second reaction chamber further contains an inhibitor of binding of the second member of the biological binding pair that specifically binds to the immobilized first member of the biological binding pair, and further comprising a substrate for the electrochemical catalyst; the method further comprising the steps of:
b) performing an electrochemical assay in each of the first and second reaction chambers of the apparatus of claim 42 to produce a current in the electrodes of the apparatus; and
c) comparing the current produced in the electrochemical assay in the first reaction chamber to the current produced in the electrochemical assay in the second reaction chamber
wherein an inhibitor of binding of the second member of the biological binding pair with the immobilized first member of the biological binding pair is identified by the production of a larger current in the first reaction chamber than is produced in the second reaction chamber.

53. A method for screening a complex chemical mixture for an inhibitor of binding of an electrochemically labeled second member of a biological binding pair with a first member of a biological binding pair immobilized on an electrode using an apparatus according to claim 42, the method comprising the steps of:
a) providing a first reaction chamber in electrochemical contact with each of the electrodes according to claim 42, wherein the first electrode comprises a first member of the biological binding pair immobilized thereto, and a second reaction chamber in electrochemical contact with each of the electrodes according to claim 42, wherein the first electrode comprises a first member of the biological binding pair immobilized thereto, each of the electrodes being electrically connected to a potentiostat;
wherein each of the reaction chambers contains a substrate for the electrochemical catalyst and a second member of the biological binding pair that specifically binds to the immobilized first member of the biological binding pair, bound to an electrochemical catalyst, and wherein the second reaction chamber further contains a portion of the complex mixture comprising an inhibitor of binding of the second member of the biological binding pair that specifically binds to the immobilized first member of the biological binding pair and further comprising a substrate for the electrochemical catalyst; the method further comprising the steps of:
b) performing an electrochemical assay in each of the first and second reaction chambers of the apparatus of claim 42 to produce a current in the electrodes of the apparatus; and
c) comparing the current produced in the electrochemical assay in the first reaction chamber to the current produced in the electrochemical assay in the second reaction chamber
wherein a complex mixture having an inhibitor of binding of the second member of the biological binding pair with the immobilized first member of the biological binding pair is identified by the production of a larger current in the first reaction chamber than is produced in the second reaction chamber.

54. A method according to claim 51 wherein the second member of the biological binding pair is a surrogate ligand.

55. A method according to claim 52 wherein the second member of the biological binding pair is a surrogate ligand.

56. A method according to claim 53 wherein the second member of the biological binding pair is a surrogate ligand.

57. A method according to claim 53 comprising the additional steps of:
d) chemically fractionating the complex mixture having an inhibitor of binding of the second member of the biological binding pair to the first member of the biological binding pair immobilized on the first electrode, to produce fractionated submixtures; and
e) performing steps (a) through (c) of the method of claim 53 on each of the fractionated submixtures to identify the submixtures that have an inhibitor of binding of the biological binding pair.

58. A method according to claim 55 wherein the inhibitor is added to the second reaction chamber after the surrogate ligand is added to the second reaction chamber.

59. A method according to claim 55 wherein the inhibitor is added to the second reaction chamber before the surrogate ligand is added to the second reaction chamber.

60. A method according to claim 55 wherein the inhibitor is added to the second reaction chamber together with the surrogate ligand is added to the second reaction chamber.
